(19) United States
(12) Patent Application Publication
(10) Pub. No.: US 2024/0280905 A1
(43) Pub. Date: Aug. 22, 2024

(54) UNDERLAYER AND METHODS FOR EUV LITHOGRAPHY

(71) Applicant: Brewer Science, Inc., Rolla, MO (US)

(72) Inventors: Si Li, Rolla, MO (US); Ming Luo, Saint Louis, MO (US); Ruimeng Zhang, Holmdel, NJ (US); Kelsey Brakensiek, Rolla, MO (US); Xue Wang, Holmdel, NJ (US); Yichen Liang, Round Rock, TX (US); Xinlin Lu, Rolla, MO (US); Pengtao Lu, Rolla, MO (US)

(21) Appl. No.: 18/440,906

(22) Filed: Feb. 13, 2024

Related U.S. Application Data
(60) Provisional application No. 63/445,213, filed on Feb. 13, 2023.

(51) Int. Cl. G03F 7/11; C07D 301/00; C07F 7/08; C08G 77/26; C08G 77/50; C09D 183/04; C09D 183/14

(52) U.S. Cl. CPC G03F 7/11; C07D 301/00; C07F 7/0834; C08G 77/26; C08G 77/50; C09D 183/04; C09D 183/14

(57) ABSTRACT

Novel lithographic compositions for use as an EUV underlayer are disclosed. The invention includes methods of fabricating microelectronics structures using those compositions as well as structures formed by those methods. The method involves utilizing an underlayer immediately below the photoresist layer. The underlayer can either be directly applied to the substrate, or it can be applied to any intermediate layer(s) that may be applied to the substrate. The preferred underlayers are formed from spin-coatable, monomeric, oligomeric, and/or polymeric compositions and exhibit uniform thicknesses and low roughness. The disclosed method enables a 14/28 nm pattern using EUV lithography and better depth of focus (DOF) than standard EUV underlayers.

### CROSS-REFERENCE TO RELATED APPLICATIONS

The present application claims the priority benefit of U.S. Provisional Patent Application Ser. No. 63/445,213, filed Feb. 13, 2023, entitled SPIN-ON PRIMER AND METHODS FOR EUV LITHOGRAPHY, the entirety of which is incorporated by reference herein.

### BACKGROUND

### Field

The present disclosure relates to materials and methods for fabricating microelectronic structures using EUV (extreme ultraviolet) lithography.

### Description of Related Art

As the semiconductor industry continues to follow Moore's law, the demand for ever-decreasing feature sizes requires the use of thinner films to prevent pattern collapse. Thinner films require using a hardmask to transfer the pattern to the substrate. Extreme ultraviolet (EUV) exposure is expected to be the method of choice for single exposure lithography to achieve the required critical dimension (CD) targets of the 7-nm node and beyond. Unfortunately, EUV lithography has been hindered by a number of problems, including a lack of powerful radiation sources, stochastic effects, and adhesion issues.

Traditional trilayer stacks, containing a carbon-containing layer, a silicon-containing underlayer, and a photoresist, often suffer from poor adhesion between the photoresist and the silicon underlayer. This poor adhesion can lead to significant collapse in the patterned resist, especially at lower critical dimensions.

One approach has been to implement spin-on silicon hardmasks that offer better adhesion to the photoresist due to their relatively high carbon content. One significant compromise to the improved adhesion is an undesirable and substantial decrease in the CF₄ etch rate as a result of the lower silicon content in the silicon hardmask (Si-HM) layer.

An alternative stack architecture utilizing a “quad-layer stack” or “tri-layer stack” has also been proposed. In this architecture, a thicker underlayer is coated between a silicon-containing layer that is on the substrate (or any intervening layers on the substrate) and the photoresist. This underlayer is intended to act as an adhesion promoter between the two layers and to prevent diffusion of photoresist components into the underlayers, however, the prior art layers have been lacking in these respects.

### SUMMARY

In one embodiment, the present disclosure is broadly concerned with a method of forming a structure. The method comprises forming an underlayer on a stack, forming a photoresist layer on the underlayer, and subjecting at least a portion of the photoresist layer to EUV radiation. The underlayer is formed from a composition comprising a component chosen from:
(a) a polymer, an oligomer, or a mixture thereof comprising at least about 75 mol % of a monomer having an adhesion moiety and a surface modifying group chosen from a silanol moiety, an Si—O moiety, or both;
(b) a monomeric compound comprising an adhesion moiety and a surface modifying group chosen from a silanol moiety, an Si—O moiety, or both; or
(c) combinations of (a) and (b).

In a further embodiment, the disclosure provides a structure comprising:
a substrate having a surface;
optionally one or more intermediate layers on the substrate surface, there being an uppermost intermediate layer on the substrate surface, if one or more intermediate layers are present;
an underlayer on the substrate surface, or on the uppermost intermediate layer; and
a photoresist on the underlayer. The underlayer comprises:
(a) a polymer, an oligomer, or a mixture thereof comprising at least about 75 mol % of a monomer having an adhesion moiety at a first end of an alkyl and an Si—O moiety at a second end of the alkyl;
(b) a monomeric compound comprising an adhesion moiety at a first end of an alkyl and an Si—O moiety at a second end of the alkyl; or
(c) combinations of (a) and (b).

In yet a further embodiment, a method of hydrolyzing a silane is provided. The method comprises combining the silane with an acid and at least one solvent and allowing the silane to hydrolyze for at least 60 minutes. The silane is chosen from glycidylpropylmethyldiethoxysilane, 2-(3,4-epoxycyclohexyl)ethylmethyldiethoxysilane, phenyltrimethoxysilane, (1,3-di-2-propen-1-yl)-5-(([3-triethoxysilylpropyl])-(1,3,5-triazine-2,4,6 (1H,3H,5H)-trione), (methacryloxymethyl)methyl-dimethoxysilane, 2-chloroethylmethyldimethoxysilane, 3-acetoxypropylmethyldimethoxysilane, 3-mercaptopropylmethyldimethoxysilane, 3-phenylpropylmethyldimethoxysilane, 4-amino-3,3-dimethylbutylmethyldimethoxysilane, vinylmethyldimethoxysilane, dimethyl-dimethoxysilane, phenylmethyldimethoxysilane, glycidylpropyltrimethoxysilane, 2-(3,4-2-(3,4-epoxycyclohexyl)ethyltrimethoxysilane, epoxy cyclohexyl)ethyltriethoxysilane, 2-chloroethyltrimethoxysilane, 3-(methacryloxy-methyl)trimethoxysilane, acetoxypropyltrimethoxysilane, 3-mercaptopropyltrimethoxysilane, 3-phenylpropyltrimethoxysilane, 4-amino-3,3-dimethylbutyltrimethoxysilane, vinyltrimethoxysilane, methyltrimethoxysilane, (3-triethoxysilyl)propyl]succinic anhydride, or combinations thereof.

### BRIEF DESCRIPTION OF THE DRAWINGS

FIG. 1 is a schematic diagram (not to scale) showing one embodiment of a lithography stack;

FIG. 2 shows the thickness uniformity of the material from Example 1;

FIG. 3 shows a line-space focus exposure matrix (FEM) of Monomeric Underlayer 1 in Example 10;

FIG. 4 is a line-space Scanning Electron Microscope (SEM) photograph (164 kX) of Monomeric Underlayer 1 in Example 10;

FIG. 5 is a Bossung plot of line-space critical dimension versus focus for Monomeric Underlayer 1 in Example 10;

FIG. 6 is a line-space FEM of Monomeric Underlayer 2 in Example 11;

FIG. 7 is a graph of critical dimension versus focus for Monomeric Underlayer 2 in Example 11;

FIG. 8 is a graph of line-width roughness versus focus for Monomeric Underlayer 2 in Example 11;

FIG. 9 is a cross-section of an SEM photograph (164 kX) of EUV lithography performed using Monomeric Underlayer 2 in Example 11;

FIG. 10 is a comparison of CD-dose of a standard EUV underlayer versus Monomeric Underlayer 2 in Example 12;

FIG. 11 is a comparison of CD-DOF of a standard EUV underlayer versus Monomeric Underlayer 2 in Example 12;

FIG. 12 is a comparison of CD-LWR of a standard EUV underlayer versus Monomeric Underlayer 2 in Example 12;

FIG. 13 is a comparison of process window of a standard EUV underlayer versus Monomeric Underlayer 2 at their best-performing bake temperature in Example 12;

FIG. 14(A) shows the influence of bake temperature of Monomeric Underlayer 2 in Example 12 on CD-dose;

FIG. 14(B) shows the influence of bake temperature of Monomeric Underlayer 2 in Example 12 on CD-DOF;

FIG. 14(C) shows the influence of bake temperature of Monomeric Underlayer 2 in Example 12 on CD-LWR;

FIG. 15 shows a contact hole FEM of Monomeric Underlayer 2 in Example 12;

FIG. 16 shows a defect review over CD for Monomeric Underlayer 2 and a standard EUV underlayer in Example 12;

FIG. 17 shows an EUV lithography result of Monomeric Underlayer 2 in Example 12;

FIG. 18 shows a Bossung plot of critical dimension versus focus for Monomeric Underlayer 2 in Example 12;

FIG. 19 shows CDU-DOF versus CD for Monomeric Underlayer 2 and a standard spin-on EUV underlayer in Example 12;

FIG. 20 shows CDU versus CD for Monomeric Underlayer 2 and a standard spin-on EUV underlayer in Example 12;

FIG. 21 is a line-space FEM of the material from Example 13 (see Example 21);

FIG. 22 is a top-down SEM photograph (164 kX) of the material from Example 13 (see Example 21);

FIG. 23 is a line-space FEM of the material from Example 13 (see Example 22);

FIG. 24 is a top-down SEM photograph (164 kX) of the material from Example 13 (see Example 22);

FIG. 25 is a line-space FEM of the material from Example 13 (see Example 23);

FIG. 26 is a top-down SEM photograph (164 kX) of the material from Example 13 (see Example 23);

FIG. 27 is a line-space FEM of the material from Example 14 (see Example 24);

FIG. 28 is a top-down SEM photograph (164 kX) of the material from Example 14 (see Example 24);

FIG. 29 is a line-space FEM of the material from Example 14 (see Example 25);

FIG. 30 is a top-down SEM photograph (164 kX) of the material from Example 14 (see Example 25);

FIG. 31 is a line-space FEM of the material from Example 17 (see Example 26);

FIG. 32 is a top-down SEM photograph (164 kX) of the material from Example 17 (see Example 26); and

FIG. 33 contains diagrams of two different lithography stacks and shows metal diffusion levels from a metal oxide resist into each of the stacks.

### DETAILED DESCRIPTION

The present disclosure is broadly concerned with compositions for forming underlayers (preferably ultrathin underlayers) and methods of using those compositions to form microelectronic structures. The compositions are useful at a wide range of wavelengths but are particularly well-suited for EUV lithography processes.

### Compositions for Underlayer

Suitable compositions for use as the underlayer described herein comprise a component chosen from one or more of a monomer, oligomer, and/or polymer. Preferred such components comprise an adhesion moiety that improves the adherence of a photoresist layer to the formed underlayer and/or “entangles” the underlayer with the photoresist layer. Preferred adhesion moieties include epoxy groups (including glycidyl and/or epoxycyclohexyl groups), isocyanurate groups, benzene groups, carbomethoxy groups, vinyl groups, anhydride groups, carboxylic acids (including dicarboxylic acids), chlorine atoms, or combinations thereof.

It is also preferred that the selected component includes one or more functional groups capable of performing surface modifications (e.g., enhancing surface wettability and/or adhesion) for coupling the underlayer to the underlying substrate or any intermediate layer, reducing scumming/footing, and/or achieving other effects that may be desired for lithographic performance. Examples of suitable such surface modifying groups include silanol moieties (e.g., a —SiR(OH)₂ group, which includes two silanol moieties, or a —Si(OH)₃ group, which includes three silanol moieties), an Si—O moiety (i.e.,

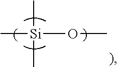

or combinations thereof.

In one or more embodiments, the monomeric compound or recurring monomer (in the instance of polymeric and/or oligomeric components) comprise at least two, and more preferably at least three total surface modifying groups. Thus, the monomeric compound and/or recurring monomer could have at least one or at least two silanol moieties, at least two or at least three Si—O moieties, or at least two or at least three surface modifying groups that are a mix of silanol moieties and Si—O moieties.

Regardless of the chosen functional groups, in some embodiments, it is preferred that the surface modifying group and the adhesion moiety be spaced apart from one another within the particular monomeric compound or recurring monomer (in the instance of polymeric and/or oligomeric components). This spacing can be accomplished by a spacer moiety, preferably one that is linear. The spacer moiety preferably has a first end and a second end that is generally opposite from the first end, with the surface modifying group being at the first end, and the adhesion moiety being at the second end. Examples of suitable spacer moieties include alkyls (preferably divalent alkyls). Preferred alkyls are C₂ to about C₈ alkyls, and more preferably about C₂ to about C₅ alkyls. The alkyls can be substituted or unsubstituted.

### 1. Monomeric Compounds for Use in Compositions

When the component for the underlayer composition is a monomeric compound (i.e., not a polymer or an oligomer), it can be purchased or synthesized, depending on the user's preferences. In some embodiments, a base or starting monomer containing the adhesion moiety and a less reactive silicon-containing group, preferably spaced apart as described previously, may be hydrolyzed in a weakly acidic solution (e.g., pKa greater than 0) to form the silanol moiety and/or Si—O moiety.

To hydrolyze the monomer, an acid is charged to a reactor in an appropriate hydrolysis solvent(s) with stirring. Hydrolysis solvents include propylene glycol monomethyl ether acetate (“PGMEA”), propylene glycol methyl ether (“PGME”), acetone, propylene glycol ethyl ether (“PGEE”), cyclohexanone, ethyl lactate, 3-methyl-1,5-pentanediol, 1,2-propylene glycol, 1,3-propylene glycol, ethylene glycol, or mixtures thereof.

Suitable acids for the hydrolysis include maleic acid, nitric acid, hydrochloric acid, acetic acid, sulfuric acid, or combinations thereof. The acid is typically added at levels of about 0.001 mol % to about 0.1 mol %, preferably about 0.005 mol % to about 0.01 mol %, with mol % being based on the total moles of monomer. The acid/solvent mixture is preferably allowed to stir for about 1 minute to about 20 minutes, and more preferably about 5 minutes to about 10 minutes.

The desired monomer(s) is then charged to the reactor. Monomer percent solids in the reaction mixture are generally about 1% to about 15%, and preferably about 2% to about 8%, based on the combined weight of the reaction mixture (including monomers, acid, and solvent(s), for example) taken as 100% by weight.

Preferred starting monomers include alkoxy silanes, and particularly dialkoxy and/or trialkoxy silanes. C₁ to C₄ dialkoxy silanes and/or C₁ to C₄ trialkoxy silanes are particularly preferred, especially those containing an adhesion moiety with or without a spacer moiety as described previously.

Examples of such starting monomers include those chosen from glycidylpropylmethyldiethoxysilane (gly DEOS), 2-(3,4-epoxycyclohexyl)ethylmethyldiethoxysilane (ECHDEOS), phenyltrimethoxysilane (PTMS), (1,3-di-2-propen-1-yl)-5-(([3-triethoxysilylpropyl])-(1,3,5-triazine-2,4,6 (1H,3H,5H)-trione) (DAICATEOS), (methacryloxymethyl)methyl-dimethoxysilane, 2-chloroethylmethyldimethoxysilane, 3-acetoxypropylmethyldimethoxysilane, 3-mercaptopropylmethyldimethoxysilane, 3-phenylpropylmethyldimethoxysilane, 4-amino-3,3-dimethylbutylmethyldimethoxysilane, vinylmethyldimethoxysilane (VDMS), dimethyl-dimethoxysilane (MDMS), phenylmethyldimethoxysilane (PDMS), or combinations thereof. In a preferred embodiment, the composition is homomonomeric meaning only one type of monomer is used.

After the monomers are charged to the reactor, the reaction mixture is typically allowed to stir for about 60 minutes to about 24 hours, and preferably about 12 hours to about 20 hours. The resulting mother liquor can be used “as is” for further formulation.

Two exemplary hydrolyzation reactions are shown in Scheme A.

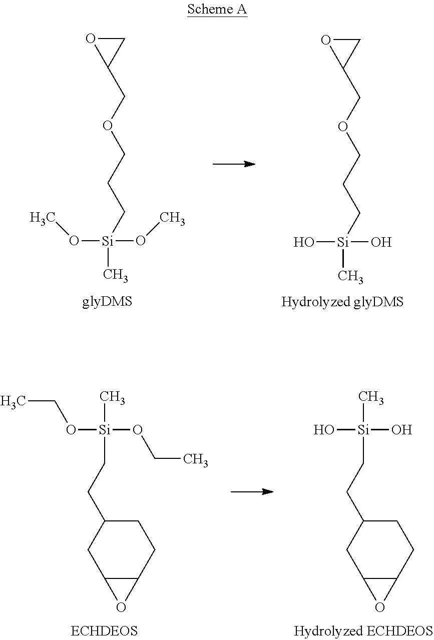

### 2. Polymers and or Oligomers for Use in Compositions

When the component for the underlayer composition is an oligomer (i.e., two to ten monomers or repeat units) or polymer, the polymer or oligomer can be purchased, or starting monomers can be polymerized or oligomerized, depending on the user's preferences. In some embodiments, a base or starting monomer containing the adhesion moiety and a less reactive silicon-containing group, preferably spaced apart as described previously, may be simultaneously polymerized and hydrolyzed in a weakly acidic solution (e.g., pKa greater than 0) to form the silanol moiety and/or Si—O moiety.

In some embodiments, the polymers and oligomers are preferably synthesized by charging the starting monomer(s) to a reactor with an optional distillation apparatus or reflux setup in an appropriate polymerization solvent with stirring. Preferred starting monomers include alkoxy silanes, and particularly trialkoxy silanes. C₁ to C₄ trialkoxy silanes are particularly preferred, especially those containing an adhesion moiety with or without a spacer moiety as described previously.

Examples of such starting monomers include those chosen from glycidylpropyltrimethoxysilane (glyTMS), 2-(3,4-epoxy cyclohexyl)ethyltriethoxysilane (ECHTEOS), 2-(3,4-epoxycyclohexyl)ethyltrimethoxysilane (ECHTMS) phenyltrimethoxysilane (PTMS), (1,3-di-2-propen-1-yl)-5-(([3-triethoxysilylpropyl])-(1,3,5-triazine-2,4,6(1H,3H,5H)-trione) (DAICATEOS), (methacryloxy-methyl)trimethoxysilane, 2-chloroethyltrimethoxysilane, 3-acetoxypropyltrimethoxysilane, 3-mercaptopropyltrimethoxysilane, 3-phenylpropyltrimethoxysilane, 4-amino-3,3-dimethylbutyltrimethoxysilane, vinyltrimethoxysilane (VTMS), methyltrimethoxysilane (MTMS), (3-triethoxysilyl)propyl]succinic anhydride (TEOSPSA), or combinations thereof.

In some embodiments, a mixture of different starting monomers can be used to form the polymer or oligomer.

In one or more embodiments, at least about 75 mol %, preferably at least about 85 mol %, more preferably at least about 95 mol % of the polymer or oligomer comprises monomers as described above. In one embodiment, the polymer and/or oligomer consists essentially of, or even consists of, one or more of the monomer types described above.

In some embodiments, the polymer is a homopolymer, and/or the oligomer is a homo-oligomer. In other words, the polymer and/or oligomer consists essentially of, or even consists of, a single type of monomer of the monomer types described above.

Polymerization solvents include PGMEA, PGME, acetone, PGEE, cyclohexanone, ethyl lactate, 3-methyl-1,5-pentanediol, 1,2-propylene glycol, 1,3-propylene glycol, ethylene glycol, or mixtures thereof. Monomer percent solids in the reaction mixture are typically about 1% to about 25%, and preferably about 5% to about 20%, based on the combined weight of the reaction mixture (including monomers, catalysts, and solvent(s), for example) taken as 100% by weight.

In some embodiments, a catalyst is then slowly charged to the reactor at a temperature of about 20° ° C. to about 30° C. and preferably about 25° C. over about 5 minutes to about 60 minutes, and preferably about 10 minutes to about 30 minutes. The reaction is typically allowed to hydrolyze for about 5 minutes to about 60 minutes, and preferably about 15 minutes to about 45 minutes.

Suitable polymerization catalysts include nitric acid, hydrochloric acid, acetic acid, trifluoroacetic acid, sulfonic acid, or combinations thereof. The catalyst is preferably added as a solution in water. The catalyst solutions are prepared in water preferably as about 0.001N to about 10N solutions, more preferably about 0.01N to about 5N solutions, and even more preferably about 3N solutions for weaker acids (i.e., pKa that is greater than 0) and about 0.01N solutions for stronger acids (i.e., pKa of about 0 or lower). These aqueous catalyst solutions are preferably added in an amount of about 0.5 equivalents to about 20 equivalents, more preferably about 5 equivalents to about 15 equivalents, and even more preferably about 10 equivalents to the total monomers, where one equivalent is approximately equal to one mole of water per mol of monomer (or to about 18 grams of water per mole of monomer).

The reaction is preferably further stirred at a temperature of about 20° C. to about 100° C., and more preferably about 40° C. to about 100° C., for about 60 minutes to about 24 hours, and preferably about 6 hours to about 16 hours. The reaction is optionally performed in an inert atmosphere such as nitrogen.

Crosslinkability of the underlayer can be enhanced during synthesis of the polymeric material by polymerizing the material under conditions that promote a more branched polymer structure rather than a more linear polymer structure. Many variables influence the polymer structure, but a more branched, higher-crosslinking polymer can be synthesized using reduced acid concentration, increasing water, increasing hydrolysis time, and/or decreasing reaction times and temperatures. These and other process conditions can be controlled to produce a higher percentage of lower-weight oligomers during synthesis, thus favoring more branching in the polymeric structure, which can lead to more crosslinking sites.

Preferred oligomers and polymers preferably have a relatively low weight-average molecular weight, i.e., preferably less than about 2,500 Daltons, more preferably less than about 2,000 Daltons, even more preferably about 300 Daltons to about 2,000 Daltons, and most preferably about 800 Daltons to about 1,500 Daltons, as determined by gel permeation chromatography (GPC) using linear polystyrene standards. Additionally, preferred oligomers and polymers preferably have a relatively low polydispersity, that is, preferably less than about 2, more preferably less than about 1.5, and even more preferably less than about 1.3.

In one or more embodiments, when the starting monomer includes 2-(3,4-epoxycyclohexyl)ethyltrimethoxysilane, either as the only monomer or as one of two or more monomer types, the polymer preferably has a weight-average molecular weight of less than about 2,000 Daltons and/or is a branched polymer.

It is also preferred in some embodiments that the polymer or oligomer backbone does not include metal atoms. In one or more embodiments, the polymer or oligomer comprises less than about 5% by weight metal, preferably less than about 3% by weight metal, more preferably less than about 1% by weight metal, and even more preferably about 0% by weight metal, based on the total weight of the polymer or oligomer taken as 100% by weight.

### 2. Composition Preparation

The reaction mixture (mother liquor) may be formulated without further processing, or the component (i.e., monomeric compound, polymer, oligomer) may be isolated before using it in a final formulation. In the latter instance, the component is dispersed or dissolved in a solvent system (i.e., one or more solvents). Preferred solvent systems include those solvents chosen from PGMEA, PGME, PGEE, propylene glycol n-propyl ether (“PnP”), ethyl lactate, cyclohexanone, gamma-butyrolactone (“GBL”), methyl isobutyl carbinol 3-methyl-1,5-pentanediol, 1,2-propylene glycol, 1,3-propylene glycol, ethylene glycol, or mixtures thereof.

The solvent system is preferably utilized at a level of about 20% to about 99.99% by weight, more preferably about 80% to 99.9%, and even more preferably about 90% to about 99.9% by weight, based upon the total weight of the composition taken as 100% by weight. The compositions used to form the underlayer preferably comprise a solids content of about 0.01% to about 20% by weight solids, more preferably about 0.01% to about 10% by weight solids, and even more preferably about 0.05% to about 1.5% by weight solids, based upon the total weight of the composition taken as 100% by weight.

The component will preferably be present at a level of about 0.01% to about 1% by weight, more preferably about 0.01% to about 0.5% by weight, even more preferably about 0.01% to about 0.1% by weight, based upon the total weight of the composition taken as 100% by weight. Additionally or alternatively, the component is preferably present at about 50% to about 100% by weight, more preferably about 98% to about 100% by weight, based upon the total weight of all solids present in the composition taken as 100% by weight.

In some embodiments, a catalyst such as a crosslinking catalyst may be included in the underlayer composition. Suitable catalysts include those chosen from thermal acid generators (TAGs, such as a quaternary ammonium blocked triflic acid thermal acid generator, e.g., TAG2689 from King Industries and TAG2690 from King Industries), acids (such as nitric acid or maleic acid), benzyltriethylammonium chloride (“BTEAC”), ethyltriphenylphosphonium bromide, tetrabutylphosphonium bromide, or combinations thereof. When used, the catalyst is preferably present in the particular composition at levels of about 0.01% to about 10% by weight, more preferably about 0.1% to about 5% by weight, based upon the total weight of the component taken as 100% by weight.

In some embodiments, a surfactant may be included in the composition to improve coating quality. Nonionic surfactants such as those having highly fluorinated alkyl groups are preferred. Commercial examples of preferred surfactants include R30N (DIC Corporation, Japan) and FS3100 (The Chemours Company FC, LLC. USA). The surfactant is preferably present in the particular composition at a level of about 0.01% to about 10% by weight, and more preferably about 0.1% to about 5% by weight, based upon the total weight of the component taken as 100% by weight.

Mixing the above ingredients together in the solvent system forms the composition that can be used to form the underlayers described below. Furthermore, any optional ingredients (e.g., surfactants) are also dispersed in the solvent system (e.g., at the same time). Examples of potential optional ingredients include those chosen from bases, base catalysts, polymers (other than, or in addition to, those described above), or mixtures thereof.

In some embodiments, the composition consists essentially of, or even consists of, the component dissolved or dispersed in the solvent system.

In other embodiments, the composition consists essentially of, or even consists of, the component and a catalyst dissolved or dispersed in the solvent system.

In one embodiment, the composition consists essentially of, or even consists of, the component and a surfactant dissolved or dispersed in the solvent system.

In yet another embodiment, the composition consists essentially of, or even consists of, the component, a catalyst, and a surfactant dissolved or dispersed in the solvent system.

In a preferred embodiment, the composition used for forming an underlayer is essentially metal-free. That is, the metal content of the composition is less than about 0.005% by weight, preferably less than about 0.001% by weight, and more preferably about 0% by weight, based upon the total weight of the solids in the composition taken as 100% by weight.

In one or more embodiments, the composition used for forming an underlayer is essentially free of chromophores that are part of the component's structure and also free of chromophores that are separately added to the composition. That is, the total chromophore content of the composition (as part of the component plus any separately added) is less than about 0.005% by weight, preferably less than about 0.001% by weight, and more preferably about 0% by weight, based upon the total weight of the solids in the composition taken as 100% by weight.

In some embodiments, the composition used for forming an underlayer is essentially free of chromophores separately added to the composition (i.e., chromophores that are not part of the component's structure). In these embodiments, the “added” chromophore content of the composition is less than about 0.005% by weight, preferably less than about 0.001% by weight, and more preferably about 0% by weight, based upon the total weight of the solids in the composition taken as 100% by weight.

### Methods of Using the Silicon Hardmask Compositions

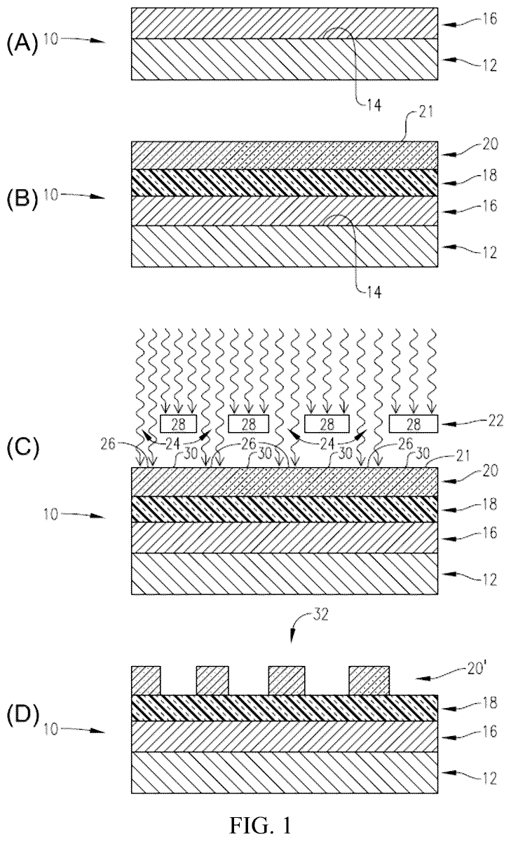

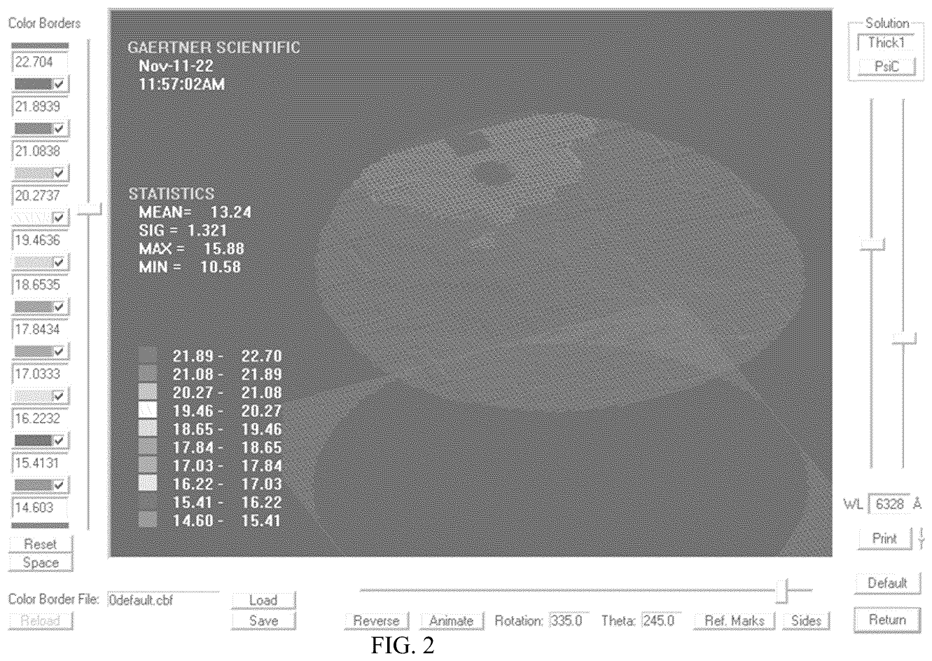

Referring to FIG. 1(A), a stack 10 is schematically depicted. Stack 10 comprises a substrate 12 having a surface 14 and optional intermediate layer(s) 16.

Substrate 12 comprises a microelectronic substrate, and preferably a semiconductor substrate. Exemplary substrates 12 comprise silicon, SiGe, SiO₂, Si₃N₄, SiON, SiCO:H (such as that sold under the name Black Diamond, by SVM, Santa Clara, CA, US), tetramethyl silate and tetramethyl-cyclotetrasiloxane combinations (such as that sold under the name CORAL), aluminum, tungsten, tungsten silicide, gallium arsenide, germanium, tantalum, tantalum nitride, Ti₃N₄, hafnium, HfO₂, ruthenium, indium phosphide, glass, or combinations of the foregoing. Surface 14 of substrate 12 can be planar, or it can include topographic features (e.g., via holes, trenches, contact holes, raised features, lines, etc.). As used herein, “topography” refers to the height or depth of a structure in or on substrate surface 14. In FIG. 1(A) the surface 14 is illustrated as having a substantially planar topography, but the topography could include non-planar topographies, such as those including lines, trenches, holes, pillars, etc.

FIG. 1 shows a single intermediate layer 16 for illustration purposes, however, the stack 10 can include multiple intermediate layers or no intermediate layers. In some embodiments, a suitable intermediate layer 16 includes a primer layer, which can include a separate and distinct layer or a layer that is more appropriately characterized as a modification of substrate surface 14. Preferred primers include hexamethyldisilizane (“HMDS”). A primer can be formed, for example, by exposing the substrate 12 to a vapor of a primer composition in a sealed chamber while heating at about 150° C. for about 90 seconds.

Another suitable intermediate layer 16 comprises a carbon-rich layer that may be formed on substrate surface 14, or on any other intermediate layer that may be present (e.g., the primed layer or modified surface as discussed above). Carbon-rich layers include spin-on carbon (SOC) layers, amorphous carbon layers, and carbon planarizing layers. Exemplary carbon-rich layers will generally be formed from a carbon-rich composition comprising a polymer dissolved or dispersed in a solvent(s), along with one or more optional ingredients, including those chosen from acid quenchers, base quenchers, catalysts, crosslinking agents, surface modification additives, or mixtures thereof. Preferred carbon-rich compositions will be capable of being formed into relatively thick layers and thus typically have a solids content of about 0.1% to about 70% by weight, more preferably about 5% to about 40% by weight, and even more preferably about 10% to about 30% by weight, based upon the total weight of the carbon-rich composition taken as 100% by weight. The term “carbon-rich” refers to compositions and/or layers comprising greater than about 50% by weight carbon, preferably greater than about 70% by weight carbon, more preferably from about 75% to about 95% by weight carbon, and even more preferably about 75% to about 80% by weight carbon, based upon the total solids in the composition taken as 100% by weight.

The carbon-rich layer can be formed by any known application method, with one preferred method being spin-coating at speeds of about 1,000 to about 5,000 rpm, and preferably about 1,250 to about 1,750 rpm, for a time period of about 30 to about 120 seconds, preferably about 45 to about 75 seconds. After the carbon-rich composition is applied, it is preferably heated to a temperature of about 100° C. to about 400° C., and more preferably about 160° C. to about 350° C., for a time period of about 30 seconds to about 120 seconds, and preferably about 45 seconds to about 60 seconds, to evaporate solvents. The average thickness of the carbon-rich layer after baking is typically about 10 nm to about 120 nm, preferably about 20 nm to about 100 nm, more preferably about 40 nm to about 60 nm, and even more preferably about 50 nm to about 60 nm. As used herein, “average thickness” is determined using an ellipsometer and taking the average of five measurements at five different locations. The carbon-rich layer may also be formed by other conventional application methods, including chemical vapor deposition (“CVD”), plasma-enhanced chemical vapor deposition (“PECVD”), atomic layer deposition (“ALD”), or plasma-enhanced atomic layer deposition (“PEALD”).

An optional hardmask layer may be applied adjacent to the carbon-rich material, to the substrate surface 14, or any intermediate layers on the substrate surface 14. The hardmask layer can be formed by any known application method, such as chemical vapor deposition (CVD) or plasma-enhanced chemical vapor deposition (PECVD). Another preferred method is spin-coating at speeds of about 1,000 to about 5,000 rpm, and preferably about 1,250 to about 1,750 rpm, for a time period of about 30 to about 120 seconds, and preferably about 45 to about 75 seconds.

Suitable hardmask layers are preferably high-silicon-content materials such as those selected from the group consisting of silanes, siloxanes, silsesquioxanes, silicon oxynitride, silicon nitride, polysilicon, amorphous silicon, or mixtures thereof, or any layer with a high etch bias relative to the underlying layers. Suitable hardmask layers generally compromise a polymer dissolved or dispersed in a solvent system, along with one or more of the following optional ingredients: surfactants, acid or base catalysts, and crosslinkers.

Preferred compositions for forming a hardmask layer preferably have a solids content of about 0.1% to about 70%, more preferably about 0.5% to about 10%, even more preferably about 0.5% to about 2% by weight, and most preferably about 0.5% to about 1% by weight, based upon the total weight of the hardmask composition taken as 100% by weight. After the hardmask is applied, it is preferably heated at a temperature of about 100° C. to about 300° C., and more preferably about 150° C. to about 250° C., for a time period of about 30 seconds to about 120 seconds, and preferably about 45 seconds to about 60 seconds, to evaporate solvents. The average thickness of the hardmask layer after baking is preferably about 5 nm to about 50,000 nm, more preferably about 5 nm to about 1,000 nm, and even more preferably about 10 nm to about 30 nm. The hardmask layer preferably has an etch rate that is about 0.75 times or higher than that of the photoresist in a fluorine-rich plasma atmosphere. Additionally or alternatively, the hardmask layer etch rate is preferably about 20% or less (five times slower) than that of the carbon-rich layer in an oxygen-rich plasma etch atmosphere.

Commercial hardmask layers can be used, with some preferred hardmask layers containing a copolymer of monomers selected from the group containing phenethyltrimethoxysilane (PETMS), 2-(carbomethoxy)ethyltrimethoxysilane (CMETMS), tetraethoxysilane (TEOS), methyltrimethoxysilane, phenyltrimethoxysilane, methyltrimethoxysilane (MTMS), ethyltrimethoxysilane (ETMS), (3-glycidyoxypropyl)triethoxysilane, and 2-(3,4-epoxycyclohexyl)ethyltrimethyoxysilane (ECHTMS).

Especially preferred silicon hardmasks have a low carbon content, preferably less than about 30% carbon, more preferably less than about 25% carbon, and even more preferably less than about 20% carbon, all by weight. The silicon hardmask preferably has high silicon content, preferably at least about 25% silicon, more preferably at least about 30% silicon, and even more preferably at least about 40% silicon, all by weight.

Regardless of whether zero, one, two, or more intermediate layers are included in stack 10, an underlayer composition as previously described can be used to form an underlayer 18 (FIG. 1(B)). Underlayer 18 can be formed directly on the substrate surface 14 if no intermediate layer is utilized (not shown), on the intermediate layer 16 if only one intermediate layer is utilized (as in FIG. 1B), or on the intermediate layer that is positioned furthest from substrate surface 14 (i.e., the uppermost intermediate layer, which is preferably a hardmask layer) in embodiments where multiple intermediate layers are included (not shown).

One preferred application method involves spin-coating the underlayer composition at speeds of about 350 rpm to about 4,000 rpm, and preferably about 1,000 rpm to about 2,000 rpm, for a time period of about 20 seconds to about 60 seconds, and preferably about 30 seconds to about 60 seconds. After the underlayer composition is applied to form underlayer 18, underlayer 18 is preferably heated at a temperature sufficiently high to evaporate substantially all (about 95% or more) and preferably all (about 100%) of the solvent present in underlayer 18 and, in some embodiments, to effect crosslinking of the polymer or oligomer in the underlayer composition. When a monomeric compound is included in the underlayer composition, preferred baking conditions preferably involve temperatures of about 120° C. to about 205° C., and more preferably about 150° C. to about 180° C., for about 20 seconds to about 60 seconds, and preferably about 30 seconds to about 60 seconds. When a polymer and/or oligomer is included in the underlayer composition, preferred baking conditions preferably involve temperatures of about 120° C. to about 250° C., and more preferably about 170° C. to about 230° C., for about 20 seconds to about 60 seconds, and preferably about 30 seconds to about 60 seconds.

Scheme B depicts the chemical reactions and/or interactions that take place during spin-coating and baking, where the component in the underlayer composition used to form underlayer 18 is the hydrolyzed glycidylpropylmethyldimethoxysilane (glyDMS) whose preparation was schematically depicted in Scheme A above.

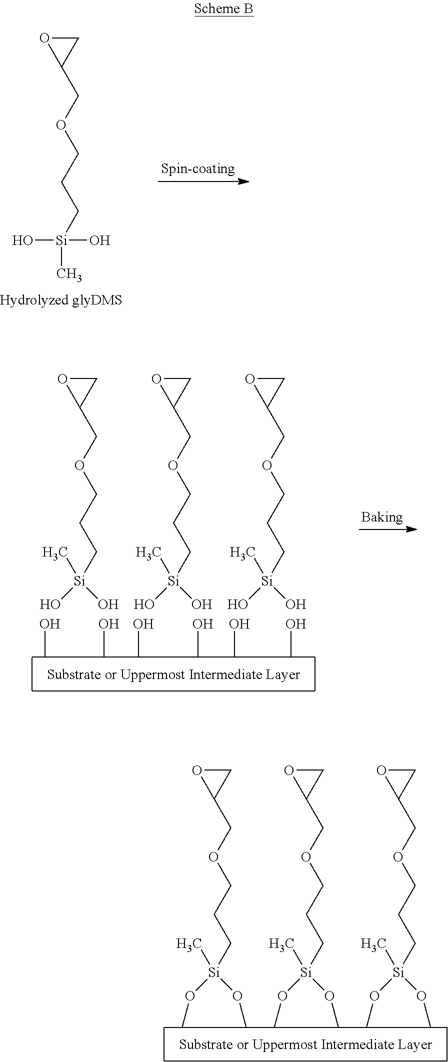

In Scheme B, the adhesion moieties comprise epoxy groups (specifically glycidyl groups), and the surface modifying groups comprise silanol moieties. The selected surface modifying groups will interact (molecular interactions and/or chemical reactions, e.g., covalent reactions) with chemical groups on the surface of the substrate or uppermost intermediate layer upon spin-coating and baking. This will cause the component of the underlayer to arrange itself such that the surface modifying groups are generally oriented toward the substrate or uppermost intermediate layer, and the adhesion moieties are generally oriented away from the substrate or uppermost intermediate layer, toward a photoresist layer that will ultimately be applied to the underlayer (as explained below).

Although Scheme B depicts this chemistry with respect to a component that is a monomeric compound (i.e., hydrolyzed glycidylpropylmethyldimethoxysilane), it will be appreciated that similar interactions would take place regardless of the selected component. Additionally, when the component or oligomer is a polymer and/or oligomer, there may be crosslinking between monomers on the same polymer or oligomer molecule and/or between different polymer or oligomer molecules.

The average thickness of the underlayer 18 after baking is less than about 4.5 nm, preferably less than about 3 nm, more preferably about 0.5 nm to 3 nm, even more preferably about 0.5 nm to about 2 nm, and most preferably about 0.5 nm to about 1 nm. In one preferred embodiment, the underlayer 18 is a monolayer. If the surface to which the underlayer 18 is applied includes topography, the underlayer 18 is preferably applied at a thickness sufficient to substantially cover the topography. In one embodiment, the underlayer 18 conforms to any topography on the surface to which the underlayer 18 is applied.

In some embodiments, the underlayer 18 has a low metal content. That is, the metal content is less than about 0.005% by weight, preferably less than about 0.001% by weight, and more preferably about 0% by weight, based upon the total weight of the underlayer 18 taken as 100% by weight. It is also preferred that the underlayer 18 is non-conducting.

It will be appreciated that the desired contact angle when a drop of water is placed on the underlayer 18 depends on the application. The surface contact angle of underlayer 18 can be determined by averaging 5 measurements taken in different spots using VCA-3000S Wafer System (AST Products, Billerica, MA) contact angle measurement tool, with water as the droplet solvent. In some embodiments, the contact angle of the underlayer 18 after baking is preferably about 50° to about 65°, and more preferably about 55° to about 59°.

It is preferred that the underlayer 18 is substantially non-developable using (i.e., substantially insoluble in) typical organic solvents such as ethyl lactate, propylene glycol methyl ether acetate, propylene glycol methyl ether, propylene glycol n-propyl ether, cyclohexanone, acetone, gamma butyrolactone, or mixtures thereof. Thus, when subjected to a stripping test, the underlayer 18 preferably has a percent stripping of less than about 25%, more preferably less than about 10%, even more preferably less than about 1%, and even more preferably about 0%. The percent stripping can be determined by measuring the average contact angle and average thickness (each determined by averaging measurements taken at five different locations) of the underlayer 18 before the underlayer 18 is exposed to any developer solvents. These averaged measurements are the initial film contact angle and initial film thickness. Next, a solvent (e.g., ethyl lactate) is puddled onto the film for about 30 seconds, followed by spin drying at about 3,000 rpm for about 30 seconds to remove the solvent. The average contact angle and average thickness are each determined again by measuring at approximately the same five locations on the wafer as the locations used to determine the initial film contact angle and initial film thickness, and the averages of these measurements are the final film contact angle and the final film thickness, respectively.

The amount of stripping is the difference between the initial and final film thicknesses. The percent stripping is:

$$\% stripping = ( \frac{amount of stripping}{initial average film thickness} ) \times 100.$$

In some cases, the underlayer 18 may be so thin that a thickness measurement is not indicative of the percent stripping. In those cases, the contact angle may be a more accurate indicator of stripping. In these cases, the same testing method is used as described previously with the thickness, except the contact change in contact angle is measured. In these cases, the variation of contact angle after stripping is preferably within about 3°, and more preferably within about 2°, and even more preferably within about 1°.

In some embodiments, the underlayer 18 is crosslinked. For example, the underlayer 18 is preferably sufficiently crosslinked that % stripping is less than about 30%, more preferably less than about 20%, and even more preferably less than about 10%. The advantages of the crosslinked nature of the underlayer 18 is that there is very little to no unbound polymer in the underlayer 18 after it is baked. This minimal or lack of unbound polymer will likely eliminate the need to strip unbound polymer during the semiconductor device manufacturing process, thereby reducing the number of steps and increasing throughput. In one embodiment, there is no solvent or developer rinse step required prior to the application of the photoresist.

In addition to the strip test, there are additional indications of a high degree of crosslinking of the underlayer 18. Preferably, there are substantially no changes in the water contact angle after contact with solvent or developer, that is, the change in contact angle is preferably less than about 5°, more preferably less than about 3°.

Another indicator of sufficient crosslinking is good thickness uniformity and a substantially uniform and defect-free surface as measured on a KLA SP5. Preferably, the underlayer 18 will have sublimation of less than about 200 ng, more preferably less than about 100 ng, and even more preferably less than about 50 ng when measured with a quartz crystal microbalance (QCM) when heated to about 205° C. for about 3 minutes. A well-crosslinked underlayer 18 will generally have a surface roughness (R1) as measured with atomic force microscopy of less than about 0.15, more preferably less than about 0.125, and even more preferably less than about 0.10.

In one or more embodiments, when the component is a polymer comprising 2-(3,4-epoxycyclohexyl)ethyltrimethoxysilane monomers, either as the only monomer or as one of two or more monomer types, the underlayer 18 is preferably crosslinked and/or exhibits less than about 30% stripping, more preferably less than about 20% stripping, even more preferably less than about 10% stripping, and most preferably less than about 2% stripping.

After the underlayer 18 is formed, a photoresist layer 20 (i.e., imaging layer) having an upper surface 21 is formed on underlayer 18. The preferred photoresist layer 20 is an EUV photoresist, and any commercial EUV photoresist composition can be utilized to form photoresist layer 20. In one embodiment, the photoresist layer 20 is a chemically amplified resist (CAR). In another embodiment, the photoresist layer 20 is a non-chemically amplified resist. In one embodiment, the non-chemically amplified photoresist includes a metal, such as those selected from the group consisting of titanium, zinc, tin, hafnium, zirconium, indium, vanadium, cobalt, molybdenum, tungsten, aluminum, gallium, silicon, germanium, phosphorous, arsenic, yttrium, lanthanum, cerium, lutetium, and mixtures of the foregoing. In another embodiment, the photoresist layer 20 comprises a metal oxide or organometallic compound in the photoresist composition.

In some embodiments, the photoresist layer 20 is substantially free of metal. That is, the metal content of the photoresist 20 is less than about 0.005% by weight, preferably less than about 0.001% by weight, and more preferably about 0% by weight, based upon the total weight of the photoresist layer 20 taken as 100% by weight.

Suitable EUV photoresists are available from several commercial suppliers including, JSR, TOK, Sumitomo, Shin Etsu, FujiFilm, Inpria, Irresistible Materials, and Zeon.

Regardless of the photoresist type, the photoresist layer 20 can be formed by any conventional method, with one preferred method being spin coating the photoresist composition at speeds of about 350 rpm to about 4,000 rpm (preferably about 1,000 rpm to about 2,500 rpm) and for a time period of about 10 seconds to about 60 seconds (preferably about 10 seconds to about 30 seconds). The photoresist layer 20 is then optionally post-application baked (“PAB”) at a temperature of at least about 45° C., preferably about 80° C. to about 250° C., and more preferably about 100° C. to about 150° C., for about 20 seconds to about 30 minutes, and more preferably about 30 seconds to about 20 minutes. The average thickness of the photoresist layer 20 after baking is typically about 5 nm to about 200 nm, preferably about 10 nm to about 50 nm, and more preferably about 20 nm to about 40 nm.

One of the advantages of using the underlayers 18 as described herein is that when used in combination with a metal oxide resist (MOR), such as some tin-oxide resists that have been developed for EUV applications, the underlayers 18 can reduce metal diffusion from the resist into the stack much more effectively than other options. This is important for minimizing or preventing the metal in the MORs from contaminating the substrate 12, intermediate layers 16, and/or the microelectronic device being manufactured. The underlayers 18 reduce metal (e.g., Sn) contamination in any intermediate layer 16 and/or substrate 12 by at least about 50%, more preferably at least about 75% when compared to the same structure but without the underlayer 18. Additionally, the underlayer 18 can prevent intermixing between the EUV photoresist layer 20 and any intermediate layers 16 under the underlayer 18.

Referring to FIG. 1(C), a mask 22 is positioned above upper surface 21 of the photoresist layer 20. The mask 22 has exposure portions 24 designed to permit the radiation to reflect from (in the case of EUV) the mask and contact the surface 21 of the photoresist layer 20, thus creating exposed areas 26 on and/or in photoresist layer 20. Mask 22 also includes nonexposure portions 28, which are designed to absorb or block the radiation to prevent the radiation from contacting surface 21 of the photoresist layer 20 in certain areas (i.e., unexposed areas 30), thus resulting in selective exposure of photoresist layer 20. Those skilled in the art will readily understand that the type of mask and the arrangement of reflecting and absorbing portions (i.e., exposure portions 24 and nonexposure portions 28) is designed based upon a desired pattern to be formed in the photoresist layer 20, and ultimately in underlayer 18, any intermediate layers 16, and the substrate 12.

The exposure wavelengths are preferably about 10 nm to about 400 nm, more preferably about 13 nm to about 193 nm. In one embodiment, the layer is exposed to wavelengths of less than about 20 nm, preferably about 11 nm to about 14 nm, and more preferably about 13.5 nm, including EUV radiation. Regardless, the preferred exposure dose is about 5 mJ/cm² to about 100 mJ/cm², preferably from about 10 mJ/cm² to about 80 mJ/cm², and more preferably from about 20 mJ/cm² to about 60 mJ/cm².

After exposure, the photoresist layer 20 is optionally subjected to a post-exposure bake (PEB) at a temperature of at least about 45° C., preferably about 60° C. to about 250° C., and more preferably about 80° C. to about 150° C., for about 20 seconds to about 5 minutes, and preferably about 30 seconds to about 2 minutes.

The photoresist layer 20 is then contacted with a developer to form a pattern 32 in the photoresist layer 20′ (FIG. 1(D)). Depending upon whether the photoresist used is positive-working or negative-working, the developer either removes the exposed portions of the photoresist layer 20′ or removes the unexposed portions of the photoresist layer 20′ to form the pattern. The pattern 32 is then transferred to the underlayer 18, any present intermediate layers 16 (e.g., hardmask layer), and finally the substrate 12. This pattern transfer can take place via plasma etching (e.g., CF₄ etchant, O₂ etchant) or a wet etching or developing process. In embodiments where the pattern is transferred from the photoresist layer 20′ to the substrate 12 via etching, it is preferred that the etch rate of the underlayer 18 relative to the EUV photoresist being used (e.g., a CAR photoresist, a non-CAR photoresist, or an organometallic photoresist) is at least about 1×, and preferably about 1.5× to about 2×.

Regardless of whether pattern transfer is effected by etching or by developing, the resulting features can have high resolutions. For example, resolutions of less than about 40 nm half pitch, and preferably less than 30 nm half pitch, can be achieved with the inventive method. Advantageously, the inventive underlayer also improves collapse margin of the final features. Collapse margin is the dose range from the dose to size and the dose at which structures are still standing.

Additional advantages of the various embodiments will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments but is not necessarily included. Thus, the present disclosure encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the disclosure. It is to be understood, however, that these examples are provided by way of illustration, and nothing therein should be taken as a limitation upon the overall scope.

### Example 1

### Synthesis and Formulation of Monomeric Underlayer 1

In this Example, 1.8 grams of water and 35.0 grams of in-house filtered PGME (Fuji Film Ultra Pure Solutions, Castroville, CA) were weighed into a 100-mL Aicello bottle and stirred using a magnetic stir bar for 5 minutes. While stirring, 0.87 gram of 0.2 wt % maleic acid in filtered PGME solution (diluted from 2 wt % maleic acid in PGME, (maleic acid from Sigma-Aldrich Inc, St. Louis, MO) was added. Next, 2.5 grams of glycidylpropylmethyldimethoxysilane (glyDMS, Gelest, Morrisville, PA) was added and stirred for an additional 5 minutes. The solution was then stirred at room temperature for another 16 hours.

The resulting mother liquor containing 5% solids in PGME was diluted to 1% solids by mixing 20 grams of the mother liquor with 70.1 grams of in-house filtered PGME and 9.9 grams of in-house filtered PGMEA (Fuji Film Ultra Pure Solutions, Castroville, CA) (90:10 mixture).

### Example 2

### Synthesis and Formulation of Monomeric Underlayer 2

In this Example, 1.8 grams of water and 35.0 grams of filtered PGME were weighed into a 100-mL Aicello bottle and stirred using a magnetic stir bar for 5 minutes. While stirring, 0.88 gram of a 0.2% maleic acid in PGME solution was added. Next, 2.58 grams of 2-(3,4-epoxycyclohexyl)ethylmethyldiethoxysilane (ECHDEOS, Gelest, Morrisville) was added and stirred for an additional 5 minutes. The solution was then stirred at room temperature for another 16 hours.

The resulting mother liquor was diluted to 1% solids in PGME/PGMEA by mixing 20 grams of the mother liquor with 70.1 grams of filtered PGME and 9.9 grams of filtered PGMEA (90:10 mixture).

### Example 3

### Synthesis and Formulation of Monomeric Underlayer 3

In this Example, 1.8 grams of water and 26.0 grams of filtered PGME were weighed into a 100-mL Aicello bottle and stirred using a magnetic stir bar for 5 minutes. While stirring, 0.65 gram of a 0.2% maleic acid in PGME solution was added. Next, 2.58 grams of phenylmethyldimethoxysilane (PDMS, Gelest, Morrisville) was added and stirred for an additional 5 minutes. The solution was then stirred at room temperature for another 16 hours.

The resulting mother liquor was diluted to 1% solids in PGME/PGMEA by mixing 20 grams of the mother liquor with 70.1 grams of filtered PGME and 9.9 grams of filtered PGMEA (90:10 mixture).

### Example 4

### Synthesis and Formulation of Monomeric Underlayer 4

In this Example, 1.8 grams of water and 24.57 grams of filtered PGME were weighed into a 100-mL Aicello bottle and stirred using a magnetic stir bar for 5 minutes. While stirring, 0.61 gram of 0.2% maleic acid in PGME solution was added. Next, 1.98 grams of phenyltrimethoxysilane (PTMS, Gelest, Morrisville) was added and stirred for an additional 5 minutes. The solution was then stirred at room temperature for another 16 hours.

The resulting mother liquor was diluted to 1% solids in PGME/PGMEA by mixing 20 grams of the mother liquor with 70.1 grams of filtered PGME and 9.9 grams of filtered PGMEA (90:10 mixture).

### Example 5

### Synthesis and Formulation of Monomeric Underlayer 5

In this Example, 1.8 grams of water and 33.89 grams of filtered PGME were weighed into a 100-mL Aicello bottle and stirred using a magnetic stir bar for 5 minutes. While stirring, 0.85 gram of 0.2% maleic acid in PGME solution was added. Next, 2.24 grams of 3-phenylpropylmethyldimethoxysilane (Gelest, Morrisville, PA) was added and stirred for an additional 5 minutes. The solution was then stirred at room temperature for another 16 hours.

The resulting mother liquor was diluted to 1% solids in PGME/PGMEA by mixing 20 grams of the mother liquor with 70.1 grams of filtered PGME and 9.9 grams of filtered PGMEA (90:10 mixture).

### Example 6

### Synthesis and Formulation of Monomeric Underlayer 6

In this Example, 1.8 grams of water and 23.31 grams of filtered PGME were weighed into a 100-mL Aicello bottle and stirred using a magnetic stir bar for 5 minutes. While stirring, 0.58 gram of 0.2% maleic acid in PGME solution was added. Next, 1.69 grams of 2-chloroethylmethyldimethoxysilane (ClDMS, Gelest, Morrisville) was added and stirred for an additional 5 minutes. The solution was then stirred at room temperature for another 16 hours.

The resulting mother liquor was diluted to 1% solids in PGME/PGMEA by mixing 20 grams of the mother liquor with 70.1 grams of filtered PGME and 9.9 grams of filtered PGMEA (90:10 mixture).

### Example 7

### Synthesis and Formulation of Monomeric Underlayer 7

In this Example, 3.6 grams of water and 28.12 grams of filtered PGME were weighed into a 100-mL Aicello bottle and stirred using a magnetic stir bar for 5 minutes. While stirring, 0.70 gram of 0.2% maleic acid in PGME solution was added. Next, 2.96 grams of dimethoxydimethylsilane (MDMS, Gelest, Morrisville) was added and stirred for an additional 5 minutes. The solution was then stirred at room temperature for another 16 hours.

The resulting mother liquor was diluted to 1% solids in PGME/PGMEA by mixing 20 grams of the mother liquor with 70.1 grams of filtered PGME and 9.9 grams of filtered PGMEA (90:10 mixture).

### Example 8

### Testing of Monomeric Underlayer 1

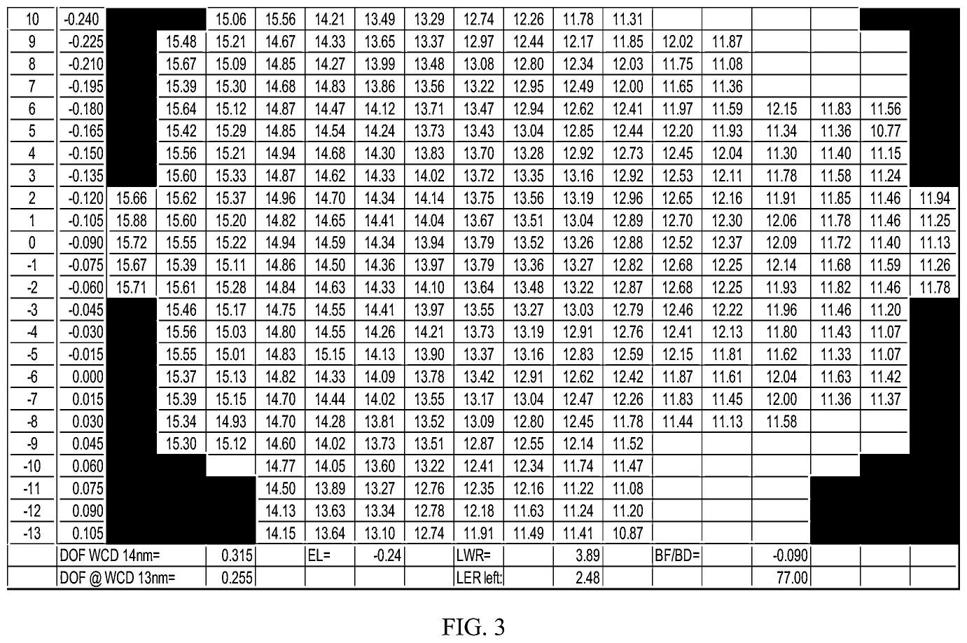

The formulation prepared in Example 1 was spin-coated on a silicon wafer at 300 rpm for 3,000 seconds and baked at 180° C. for 60 seconds. After baking, a layer a thickness of ˜10 Å was formed on the surface of silicon wafer. The coating quality was inspected by a laser confocal microscope, the uniformity was evaluated by ellipsometry, and the surface roughness was measured using atomic force microscopy. FIG. 3 shows the thickness uniformity measurements. The mean thickness was 13.24 Å and the standard deviation was 1.321 Å.

### Example 9

### Testing of Monomeric Underlayer Materials

The formulations prepared in Examples 1-7 were spin-coated on 100-mm silicon wafers at 1500 rpm for 60 seconds and baked at 180° C. for 60 seconds. Underlayer stability was evaluated using a solvent stripping test, where PGME was puddled on the underlayer for 30 seconds followed by a spin-dry at 1500 rpm. The underlayer thicknesses before and after stripping were measure measured using M-2000 Ellipsometer (J.A. Woollam Co., Lincoln, NE).

Surface energy of the underlayer was measured using AST optima (B5RM5208-143) contact angle measurement tool.

Molecular weight of each formulation was measured using gel permeation chromatography (GPC). More specifically, GPC was run using a column set as follows: Phenogel 7.8×300 mm single pore size column, 1×500 Angstrom, 1×100 Angstrom, 2×50 Angstrom, with a guard column. The column oven was set to 40° C. A mobile phase of THF (tetrahydrofuran) was used, with a flow rate of 1 mL/min. A differential refractometer was used as the detection method. Standard of polystyrene Agilent PS-L vials were used. In preparation of the formulation samples for GPC analysis, the samples were diluted to 0.5% solids in mobile phase, then an injection volume of 50 uL of each sample was used and run for 45 minutes. Results for each formulation sample are shown in Table 1.

TABLE 1
Molecular weight, thickness, and contact
angle of underlayer materials.

| | | | | H₂O | H₂O | CH₃I | CH₃I |
|---|---|---|---|---|---|---|---|
| | | Thick- | Thick- | con- | con- | con- | con- |
| | | ness | ness | tact | tact | tact | tact |
| | Molec- | after | after | angle | angle | angle | angle |
| Exam- | ular | bake | strip | after | after | after | after |
| ple | weight | (Å) | (Å) | bake | strip | bake | strip |
| 1 | 261 | 24 | 33 | 66 | 70 | 45 | 45 |
| 2 | 304 | 15 | 14 | 56.7 | 51.5 | 41.8 | 39.8 |
| 3 | 218 | 9 | 9 | 30 | 15 | 39 | 36 |
| 4 | 259 | Bad coat | N/A | N/A | N/A | N/A | N/A |
| 5 | 303 | 15 | 14 | 65 | 61 | 45 | 46 |
| 6 | 387 | 11.3 | 10.8 | 49.2 | 29.7 | 45.9 | 39.9 |
| 7 | 283 | 10 | 10 | 44 | 37 | 53 | 44 |

### Example 10

### Lithography Testing of Monomeric Underlayer 1

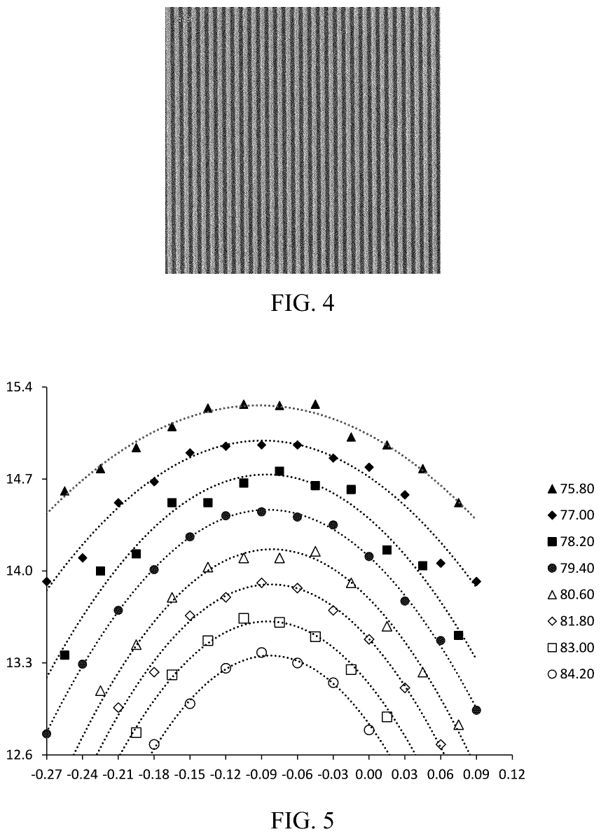

The formulation prepared in Example 1 was spin-coated as an underlayer on a prebaked silicon wafer at 1,350 rpm for 60 seconds and baked at 180° C. for 60 seconds. TOK Penta resist was then spin-coated onto the underlayer-coated wafer at 1,500 rpm for 60 seconds and baked at 110° C. for 60 seconds. EUV lithography was performed at IMEC using a NXE3400 scanner system. FIG. 4 shows EUV lithography focus exposure matrix (FEM) and scanning electron microscope (SEM) image at center dose/center focus. FIG. 5 shows a Bossong plot of CD versus focus.

### Example 11

### Lithography Testing of Monomeric Underlayer 1

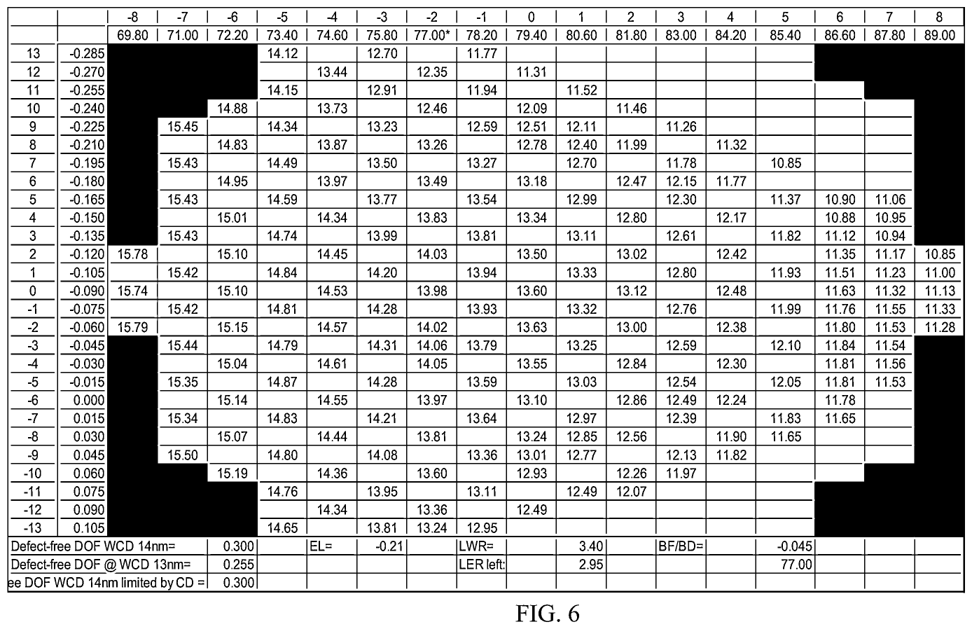

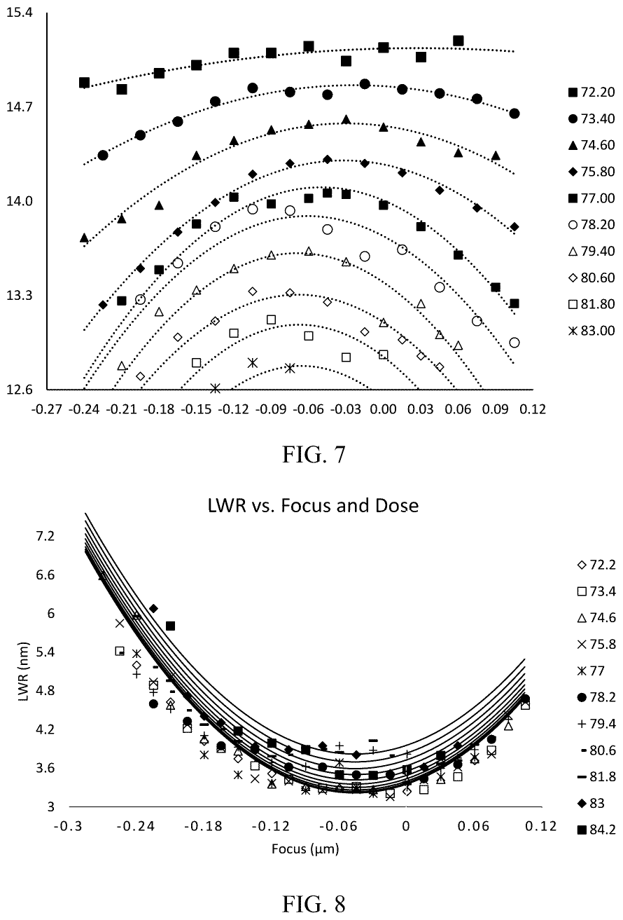

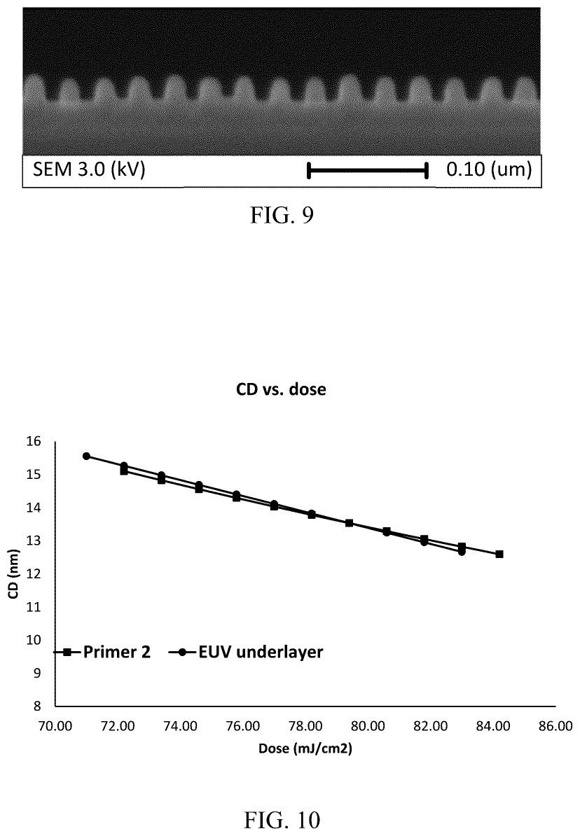

The formulation prepared in Example 2 was spin-coated on a 100-mm virgin silicon wafer prebaked at 400° C. at 1350 rpm for 60 seconds and baked at 180° C. for 60 seconds, forming an underlayer with a thickness of less than 1 nanometer with uniform thickness and low surface roughness. TOK Penta resist was then spin-coated onto the underlayer-coated wafer at 1500 rpm for 60 seconds and baked at 110° C. for 60 seconds. EUV lithography was performed at IMEC using a NXE3400 scanner system. FIG. 6 shows a line-space focus exposure matrix (FEM) for g Underlayer Material 2. FIG. 7 shows a plot of critical dimension (CD) versus focus. FIG. 8 shows a plot of line width roughness (LWR) versus focus. FIG. 9 shows a cross-section scanning electron microscope (SEM) image of the final features at the best dose and focus at 500 kx magnification.

### Example 11

### Comparison of Monomeric Underlayer 2 to Standard Spin-on EUV Underlayer

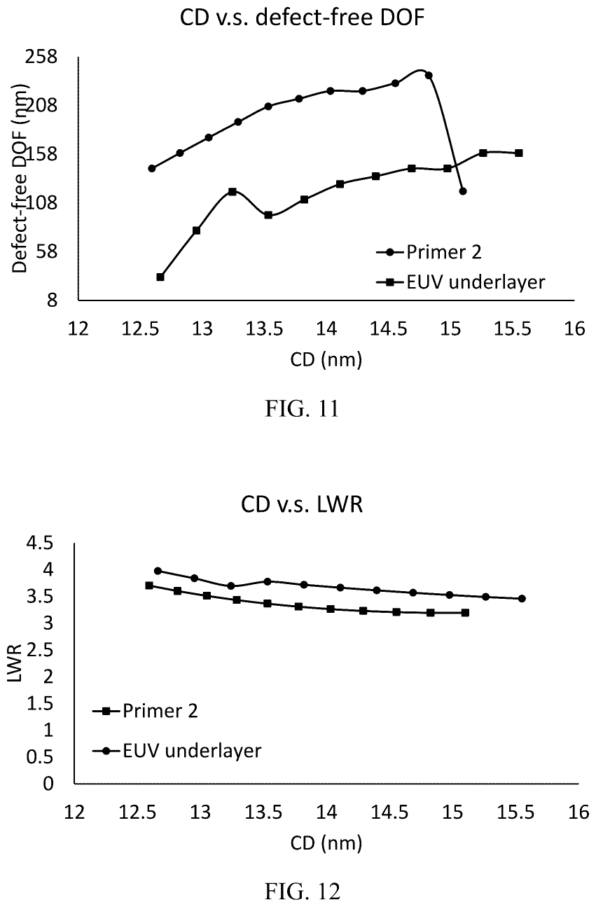

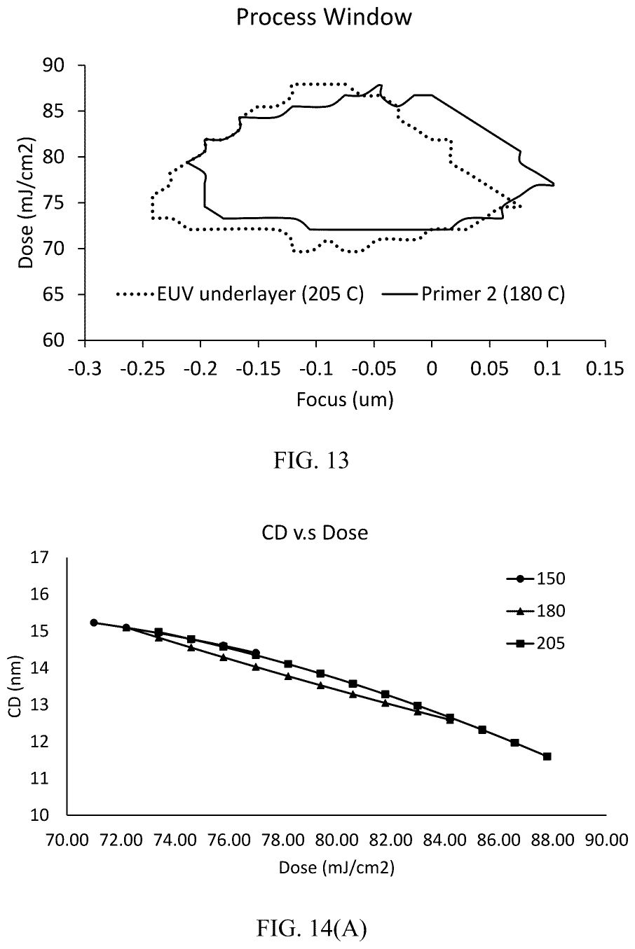

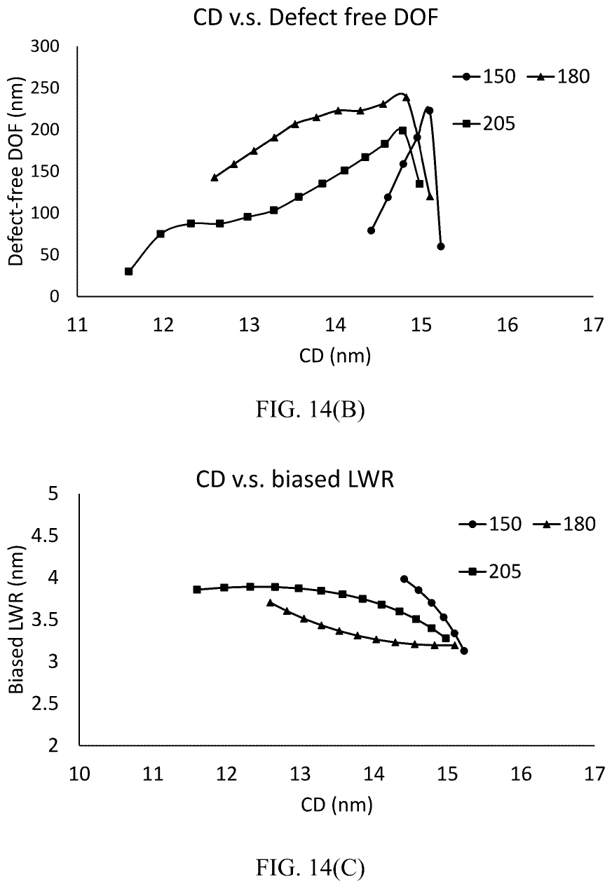

Compared to a standard spin-on silicon-containing EUV underlayer as described in U.S. Pat. No. 11,361,967, Brewer Science, Rolla, MO), the Monomeric Underlayer 2 applied as described in Example 11 showed a large process window, drastic increase in depth of focus (DOF), and reduction in LWR at similar dose. Defect-free depth of focus (DOF), the range of focus that keeps the resist profile of given features without nanobridging or line-break, was defined as assembling of dies with biased roughness (LWR) <4 nm. EUV lithography was performed at IMEC using a NXE3400 scanner system. FIG. 8 shows a comparison of dose versus CD for the two materials. FIG. 11 shows the defect-free DOF versus CD for the two materials. FIG. 12 shows a comparison of line width roughness (LWR) versus CD for the two materials. FIG. 13 shows the lithography performance of Monomeric Underlayer 2 at different bake temperatures. As shown in FIGS. 14(A)-(C), the lithography performance of Monomeric Underlayer 2 shows a strong dependence on baking temperature, wherein Monomeric Underlayer 2 demonstrates the best performance (highest DOF and lowest LWR) at 180° C.

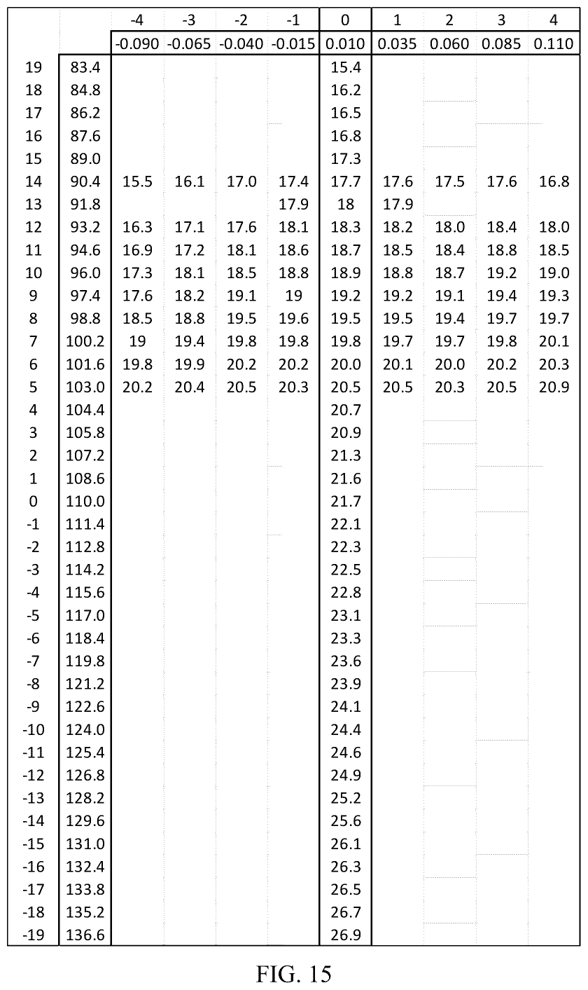

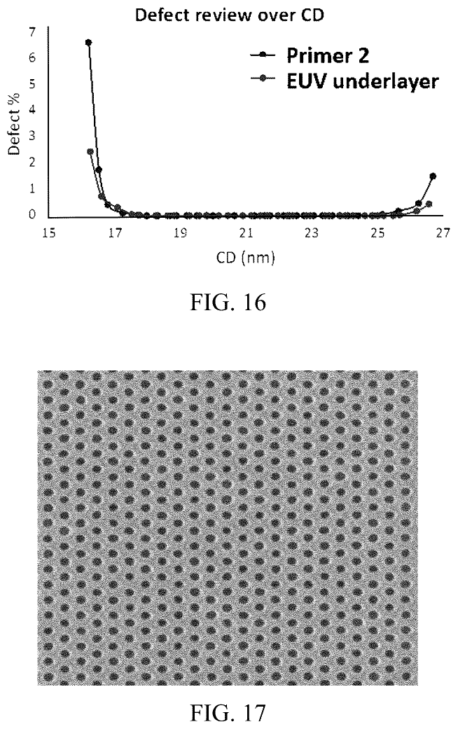

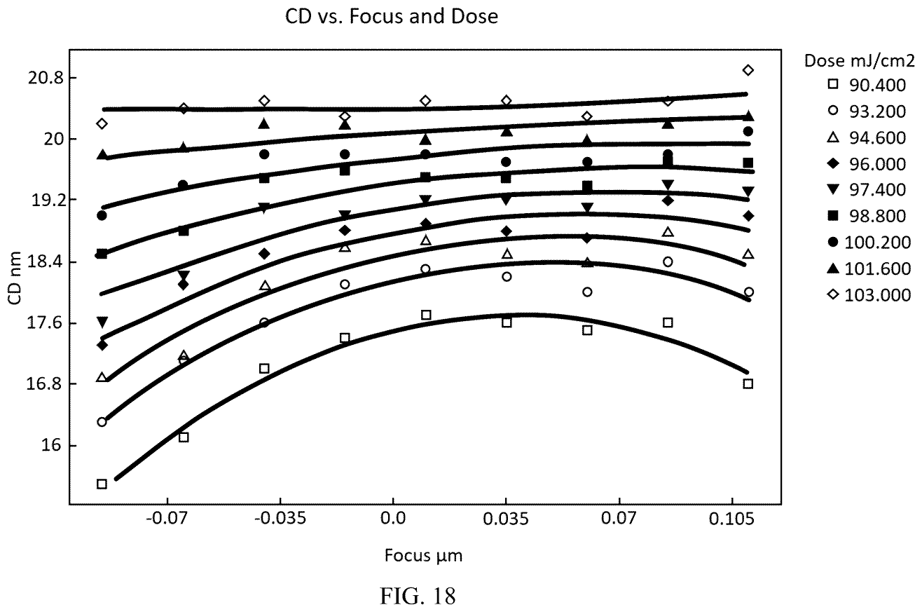

Monomeric Underlayer 2 was used to pattern contact holes in the CD range of 15.4 to 23.3 nm. FIG. 15 shows a contact hole FEM of Monomeric Underlayer 2. Kolona comparisons were conducted to give a defect review by measuring 19 CD-SEM images/chip (8600 contact holes were measured in total), where the percentage of defective contact holes were plotted versus CD. As shown in FIG. 16, Monomeric Underlayer 2 gives a similar defect-free range (defect %=0) compared to the conventional EUV underlayer, indicating a comparable large process window of Monomeric Underlayer 2. FIG. 17 shows the EUV lithography result of Monomeric Underlayer 2 for contact holes. FIG. 18 shows a Bossong plot of contact hole CD versus focus for Monomeric Underlayer 2.

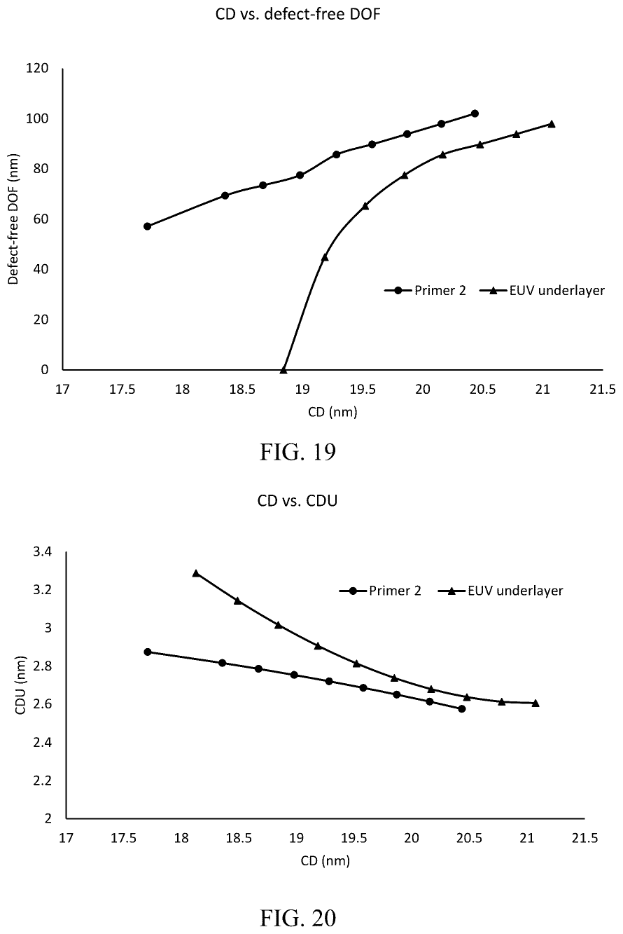

Compared to the standard spin-on underlayer thin film, Monomeric Underlayer 2 showed a large increase in DOF, and much lower critical dimension uniformity (CDU). FIG. 19 shows a comparison of Monomeric Underlayer 2 and the standard spin-on EUV underlayer in terms of CDU-DOF versus CD, where CDU-DOF was defined as the assembling of dies with CDU <3.5 nm. FIG. 20 shows a comparison of Monomeric Underlayer 2 and the standard spin-on underlayer in terms of CDU versus CD, where the unbiased 3 sigma LCDU was 1.932 as measured by Metro LER.

### Example 12

### Synthesis of Polymer 1 Mother Liquor

In this Example, a three-neck round-bottomed flask was charged with 12.3 grams of 2-(3,4-Epoxycyclohexyl)ethyltrimethoxysilane (ECHTMS) and 37.7 grams of PGME. The contents were stirred for 5 minutes to ensure a well-mixed solution. Then, 9 grams of 0.01 M nitric acid (HNO₃ aq) were added dropwise over a period of 15 minutes while stirring at room temperature. The contents were allowed to continue hydrolyzation for another 45 minutes. Then the contents were stirred at 300 rpm at 90° C. for 11 hours.

### Example 13

Formulation of Polymeric Underlayer 1 from Polymer 1 Mother Liquor

In this Example, 0.339 gram of the Polymer 1 Mother Liquor from Example 12 was loaded into a 100 mL Aicello bottle with 89.666 grams of filtered PGME, and 9.995 grams of filtered PGMEA, which were mixed overnight on a roller at room temperature. Then the material was filtered through a 0.1-μm filter. The resulting underlayer-forming composition is suitable for spin coating an (<2.5 nm) adhesion-promoting layer as described above and consists of the following polymer dissolved or dispersed in a solvent system.

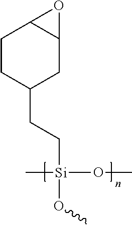

### Example 14

Formulation of Polymeric Underlayer 2 from Polymer 1 Mother Liquor

In this Example, 0.3271 gram of the Polymer 1 Mother Liquor from Example 12 was loaded into a 100 mL Aicello bottle with 0.1244 gram of 0.2% solution of R30N in PGME, 89.553 grams of filtered PGME, and 9.995 grams of filtered PGMEA, which were mixed overnight on a roller at room temperature. Then the material was filtered through a 0.1-μm filter. The resulting underlayer-forming composition is suitable for spin coating an (<2.5 nm) adhesion-promoting layer as described above and consists of the following polymer dissolved or dispersed in a solvent system.

### Example 15

Formulation of Polymeric Underlayer 3 from Polymer 1 Mother Liquor

In this Example, 0.3255 gram of the Polymer 1 Mother Liquor from Example 12 was loaded into a 100 mL Aicello bottle with 0.1980 gram of a 0.25% solution of TAG2689 in PGME, 89.482 grams of filtered PGME, and 9.995 grams of filtered PGMEA, which were mixed overnight on a roller at room temperature. Then the material was filtered through a 0.1-μm filter. The resulting underlayer-forming composition is suitable for spin coating an (<2.5 nm) adhesion-promoting layer as described above and consists of the following polymer dissolved or dispersed in a solvent system.

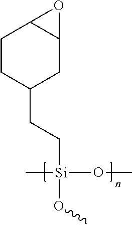

### Example 16

### Synthesis of Polymer 2 Mother Liquor

In this Example, a three-neck round-bottom flask was charged with 15.00 grams of (3-triethoxysilyl)propylsuccinic anhydride (TEOSPSA) (Gelest, Inc.) and 76.12 grams of filtered PGME. The contents were stirred for 5 minutes to ensure a well-mixed solution. Then, 8.88 grams of 0.01M HNO₃ (aq) were added dropwise over a period of 15 minutes while stirring at room temperature. The contents were allowed to continue hydrolyzation for another 45 minutes. Then the contents were stirred at 300 rpm at 90° C. for 5.5 hours under N₂ flow.

### Example 17

Formulation of Polymeric Underlayer 4 from Polymer 2 Mother Liquor

In this Example, 0.39 gram of the Polymer 2 Mother Liquor from Example 16 was loaded into an Aicello bottle with 89.41 grams of filtered PGME, 9.99 grams of filtered PGMEA, 0.06 gram of 2% maleic acid in PGME, and 0.15 gram of 0.2% ethyltriphenylphosphonium bromide (EtPPB) in PGME, which were mixed for an hour on roller at room temperature. Then the material was filtered through a 0.1-μm filter. The resulting underlayer-forming composition is suitable for spin coating a (<2.5 nm) adhesion-promoting underlayer as described above and included the following polymer dissolved or dispersed in a solvent system.

### Example 18

Formulation of Polymeric Underlayer 5 from Polymer 2 Mother Liquor

In this Example, 2.6547 grams of the Polymer 2 Mother Liquor from Example 16 were loaded into a 100-mL Aicello bottle with 1.9972 grams of a 0.2% solution of EtPPB (Ethyltriphenylphosphonium bromide) in PGME, 0.3950 grams of a 2.0% solution of maleic acid in PGME, 179.480 grams of filtered PGME, and 21.210 grams of filtered PGMEA, which were mixed overnight on a roller at room temperature. Then the material was filtered through a 0.1-μm filter. The resulting underlayer-forming composition is suitable for spin coating an (<2.5 nm) adhesion-promoting layer as described above and consists of the following polymer dissolved or dispersed in a solvent system.

### Example 19

### Synthesis of Polymer 3 Mother Liquor

In this Example, a three-neck round-bottom flask was charged with 36.92 grams of (1,3-Di-2-propen-1-yl)-5-(([3-triethoxysilylpropyl])-(1,3,5-triazine-2,4,6 (1H,3H,5H)-trione) (DAICATEOS) (Silar Laboratories), and 46.98 grams of filtered PGME. The contents were stirred for 5 minutes to ensure a well-mixed solution. Then, 16.09 grams of 0.01M HNO₃ (aq) were added dropwise over a period of 15 minutes while stirring under room temperature. The contents were allowed to continue hydrolyzation for another 45 minutes. Then the contents were stirred at 300 rpm at 90° C. for 11 hours under N₂ flow.

### Example 20

Formulation of Polymeric Underlayer 6 from Polymer 3 Mother Liquor

In this Example, 0.27 gram of the Polymer 3 Mother Liquor from Example 19 was loaded into an Aicello bottle with 134.48 grams of filtered PGME, 14.99 grams of filtered PGMEA, 0.07 gram of 2% maleic acid in PGME, and 0.18 gram of 0.2% ethyltriphenylphosphonium bromide (EtPPB) in PGME, which were mixed for an hour on a roller at room temperature. Then the material was filtered through a 0.1-μm filter. The resulting underlayer-forming composition is suitable for spin coating a (<2.5 nm) adhesion-promoting underlayer as described above and consists of the following polymer dissolved or dispersed in a solvent system.

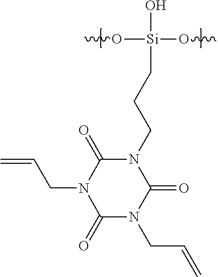

### Example 21

### Lithography Testing of Polymeric Underlayer 1 on CAR Resist

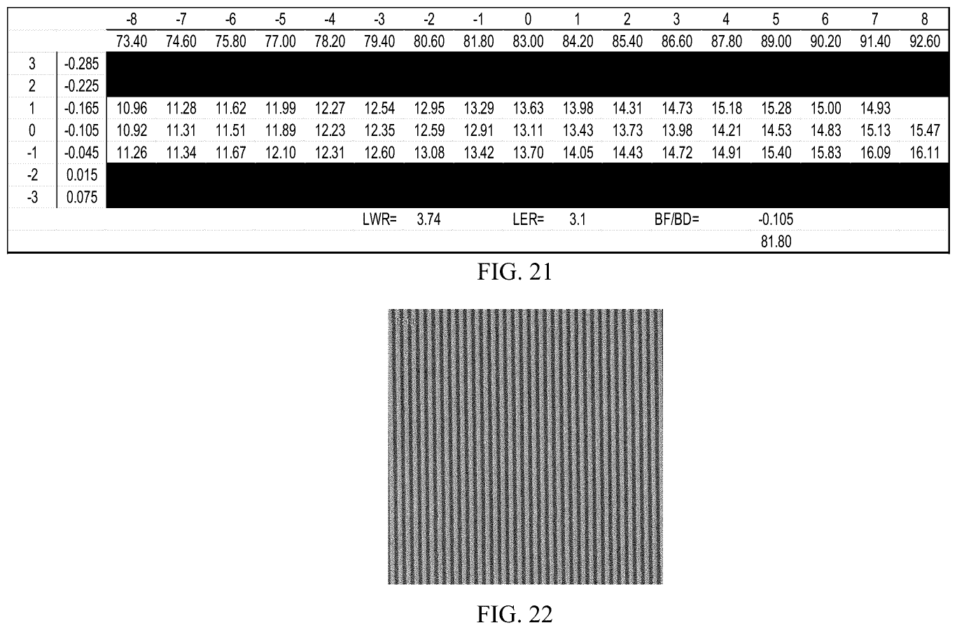

A bare silicon wafer was primed using hexamethyldisilazane (HMDS) and baked at 135° C. Following priming treatment of the wafer, an experimental silicon hard mask (“SiHM”) from Brewer Science, Inc. was spin-coated on the wafer at 1,391 rpm for 60 seconds and baked at 205° C. for 60 seconds, targeting a 10-nm film. The formulation prepared in Example 13 was then spin-coated as an underlayer on the experimental hardmask at 1,654 rpm for 60 seconds and baked at 205° C. for 60 seconds, targeting an underlayer with thickness of less than 2.5 nanometers. TOK Penta resist was then spin-coated onto the primer-coated wafer at 1,500 rpm for 60 seconds and baked at 110° C. for 60 seconds. EUV lithography was performed at IMEC using a NXE3400 scanner system. FIG. 21 shows EUV lithography focus exposure matrix (FEM), and FIG. 22 shows a scanning electron microscope (SEM) image at center dose/center focus.

### Example 22

### Lithography Testing of Polymeric Underlayer 1 on CAR Resist

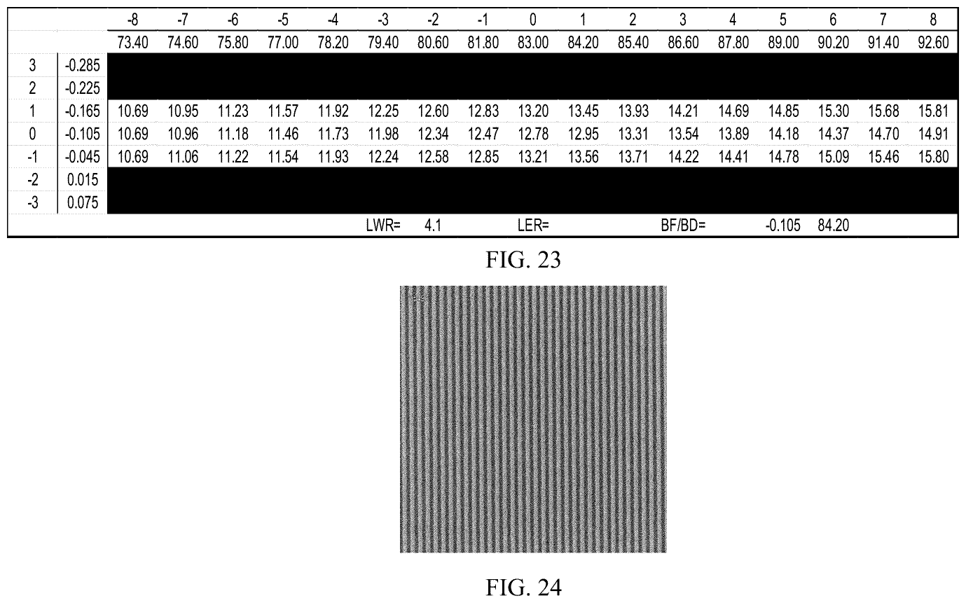

The formulation prepared in Example 13 was spin-coated on a 100-mm virgin (untreated) silicon wafer at 1,983 rpm for 60 seconds and baked at 205° C. for 60 seconds, forming an underlayer with a thickness of less than 2.5 nanometers with uniform thickness and low surface roughness. TOK Penta resist was then spin-coated onto the primer-coated wafer at 1,500 rpm for 60 seconds and baked at 110° C. for 60 seconds. EUV lithography was performed at IMEC using a NXE3400 scanner system. FIG. 23 shows EUV lithography focus exposure matrix (FEM) and FIG. 24 shows a scanning electron microscope (SEM) image at center dose/center focus.

### Example 23

### Lithography Testing of Polymeric Underlayer 1 on MOR Resist

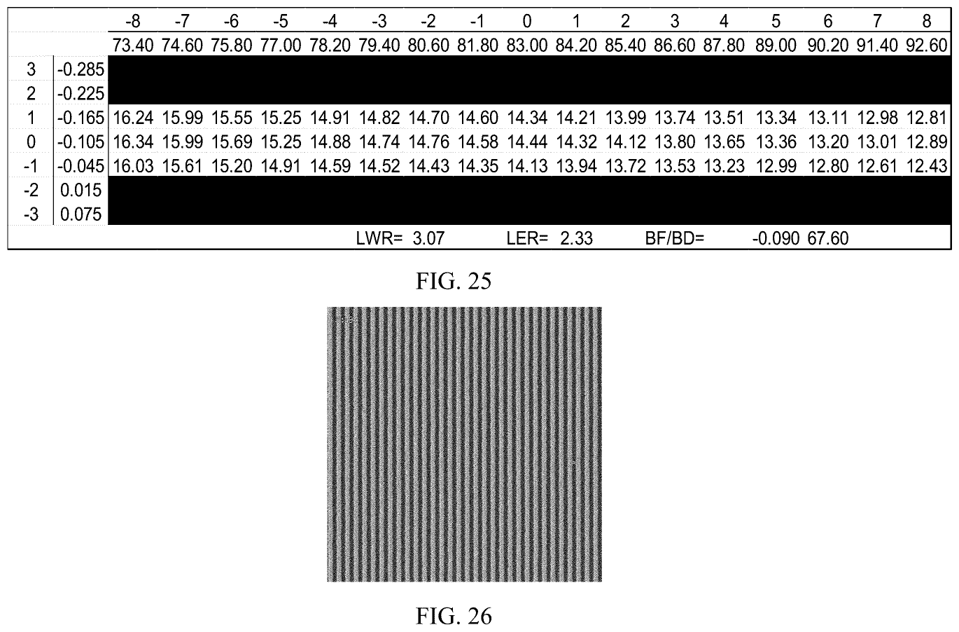

The formulation prepared in Example 13 was spin-coated on a 100-mm virgin (untreated) silicon wafer at 1,983 rpm for 60 seconds and baked at 205° C. for 60 seconds, forming an underlayer with a thickness of less than 2.5 nanometers with uniform thickness and low surface roughness. Inpria YATU1011 resist was coated at 1,500 rpm for 60 seconds and baked at 100° C. for 60 seconds. EUV lithography was performed at IMEC using a NXE3400 scanner system. FIG. 25 shows EUV lithography focus exposure matrix (FEM) and FIG. 26 shows a scanning electron microscope (SEM) image at center dose/center focus.

### Example 24

### Lithography Testing of Polymeric Underlayer 2 on CAR Resist

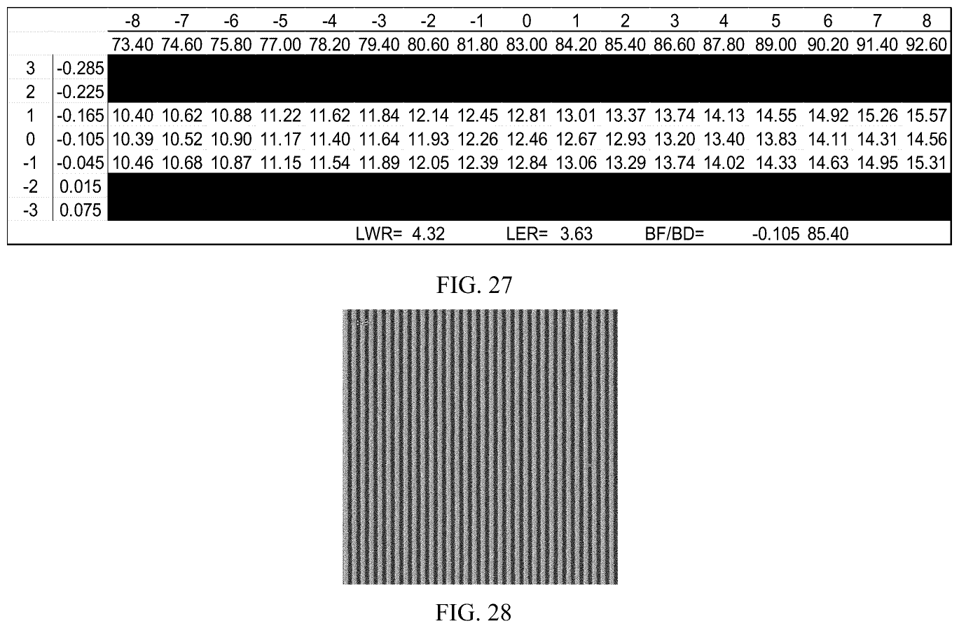

The formulation prepared in Example 14 was spin-coated on a 100-mm virgin (untreated) silicon wafer at 1,983 rpm for 60 seconds and baked at 205° C. for 60 seconds, forming an underlayer with a thickness of less than 2.5 nanometers with uniform thickness and low surface roughness. TOK Penta resist was then spin-coated onto the primer-coated wafer at 1,500 rpm for 60 seconds and baked at 110° C. for 60 seconds. EUV lithography was performed at IMEC using a NXE3400 scanner system. FIG. 27 shows EUV lithography focus exposure matrix (FEM) and FIG. 28 shows a scanning electron microscope (SEM) image at center dose/center focus.

### Example 25

### Lithography Testing of Polymeric Underlayer 2 on CAR Resist

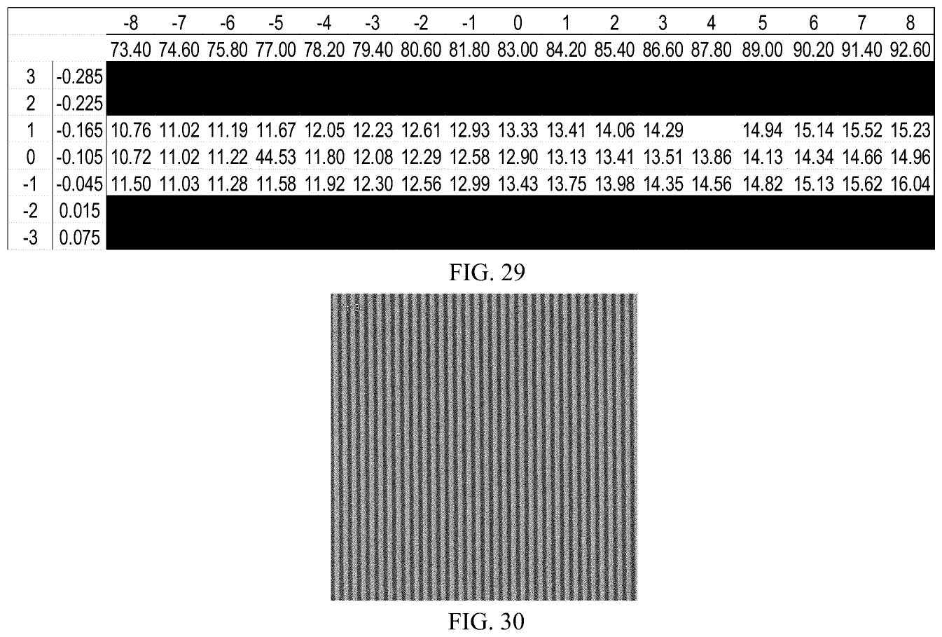

An experimental silicon hard mask (“SiHM”) from Brewer Science, Inc. was spin-coated on a bare (untreated) silicon wafer at 1,391 rpm for 60 seconds and baked at 205° C. for 60 seconds, targeting a 10-nm film. The formulation prepared in Example 14 was then spin-coated as an underlayer on the experimental hard mask at 1,654 rpm for 60 seconds and baked at 205° C. for 60 seconds, targeting an underlayer with thickness of less than 2.5 nanometers. TOK Penta resist was then spin-coated onto the -coated wafer at 1,500 rpm for 60 seconds and baked at 110° C. for 60 seconds. EUV lithography was performed at IMEC using a NXE3400 scanner system. FIG. 29 shows EUV lithography focus exposure matrix (FEM) and FIG. 30 shows a scanning electron microscope (SEM) image at center dose/center focus.

### Example 26

### Lithography Testing of Polymeric Underlayer 4 on MOR Resist

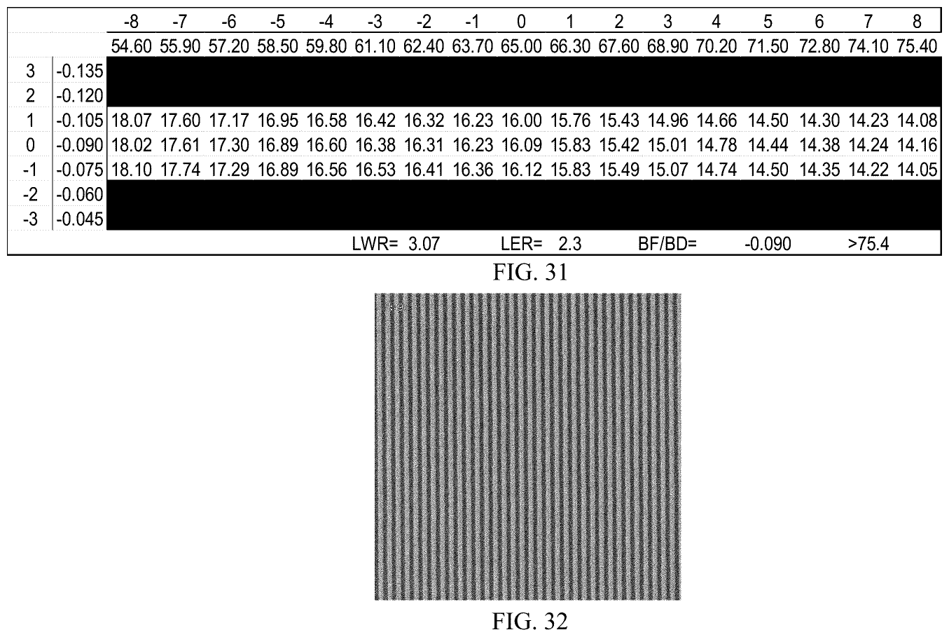

The formulation prepared in Example 17 was spin-coated on a 100-mm virgin (untreated) silicon wafer at 1,500 rpm for 60 seconds and baked at 205° C. for 60 seconds, forming an underlayer with a thickness of less than 2.5 nanometers with uniform thickness and low surface roughness. Inpria YATU1011 Resist was coated at 1,500 rpm for 60 seconds and baked at 100° C. for 60 seconds. EUV lithography was performed at IMEC using a NXE3400 scanner system. FIG. 31 shows EUV lithography focus exposure matrix (FEM), and FIG. 32 shows a scanning electron microscope (SEM) image at center dose/center focus.

### Example 27

Metal Diffusion into Underlayer Stack

An experimental silicon hard mask (“SiHM”) from Brewer Science, Inc. was spin-coated on a wafer at 1,500 rpm for 60 seconds and baked at 205° C. for 60 seconds, targeting a 10-nm film. The formulation prepared in Example 13 was then spin-coated as an underlayer on the experimental hardmask at 1,500 rpm for 60 seconds and baked at 205° C. for 60 seconds, targeting an underlayer with thickness of less than 2 nanometers. Inpria YATU1011 resist was then spin-coated onto the primer-coated wafer at 1,500 rpm for 30 seconds and baked at 100° C. for 60 seconds, followed by a bake at 180° C. for 60 seconds.

An experimental silicon hard mask (“SiHM”) from Brewer Science, Inc. was spin-coated on a control wafer at 1,500 rpm for 60 seconds and baked at 205° C. for 60 seconds, targeting a 10-nm film. Without applying an underlayer as described herein, Inpria YATU1011 resist was then spin-coated onto the primer-coated wafer at 1,500 rpm for 30 seconds and baked at 100° C. for 60 seconds, followed by a bake at 180° C. for 60 seconds.

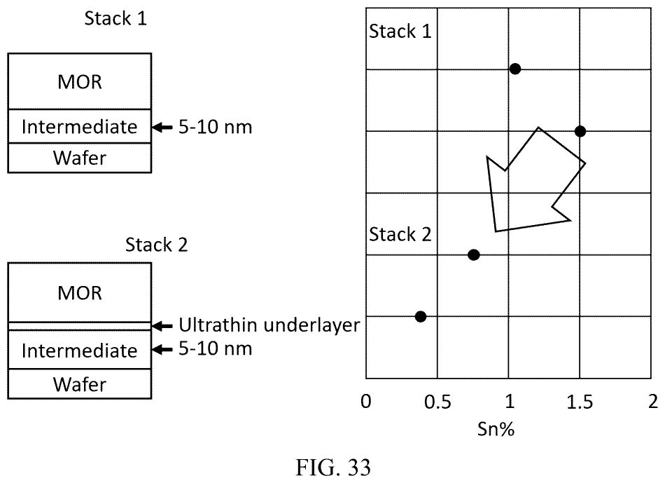

Both wafers were subjected to a developing step involving puddling IDS-033-AA developer (Inpria Corporation, Corallis, OR) for 20 seconds, followed by a 5-second rinse, and then spun dry for 45 seconds to remove the resist. The wafers were then baked at 250° C. for 60 seconds. Both wafers were then subjected to XPS analysis of the under Si-HM layer surface (for Stack 1) and underlayer (for Stack 2). As shown in FIG. 33, when used, the underlayers reduce Sn contamination in the surface layer by at least about 25%, and up to about 75%. Additionally, the underlayer can prevent intermixing between the EUV photoresist layer and any intermediate layers under the underlayer.

1. A method of forming a structure, said method comprising:
forming an underlayer on a stack, said underlayer formed from a composition comprising a component chosen from:
(a) a polymer, an oligomer, or a mixture thereof comprising at least about 75 mol % of a monomer having an adhesion moiety and a surface modifying group chosen from a silanol moiety, an Si—O moiety, or both;
(b) a monomeric compound comprising an adhesion moiety and a surface modifying group chosen from a silanol moiety, an Si—O moiety, or both; or
(c) combinations of (a) and (b); and
forming a photoresist layer on said underlayer; and
subjecting at least a portion of said photoresist layer to EUV radiation.

2. The method of claim 1, wherein:
(i) said monomer further comprises an alkyl having first and second ends, said surface modifying group being at said first end and said adhesion moiety being at said second end;
(ii) said monomeric compound further comprises an alkyl having first and second ends, said surface modifying group being at said first end and said adhesion moiety being at said second end; or
(iii) both (i) and (ii).

3. The method of claim 1, wherein said adhesion moiety comprises one or more of an epoxy group, an isocyanurate group, a benzene group, a carbomethoxy group, a vinyl group, an anhydride group, a carboxylic acid group, a chlorine atom, or mixtures thereof.

4. The method of claim 1, wherein:
(I) said monomer comprises at least two surface modifying groups;
(II) said monomeric compound comprises at least two surface modifying groups; or
(III) both (I) and (II).

5. The method of claim 1, wherein:
(A) said monomer comprises a hydrolyzed form of glycidylpropyltrimethoxysilane, 2-(3,4-epoxycyclohexyl)ethyltriethoxysilane, 2-(3,4-epoxycyclohexyl)ethyltrimethoxysilane phenyltrimethoxysilane, (1,3-di-2-propen-1-yl)-5-(([3-triethoxysilylpropyl])-(1,3,5-triazine-2,4,6 (1H,3H,5H)-trione), (methacryloxy-methyl)trimethoxysilane, 2-chloroethyltrimethoxysilane, 3-acetoxypropyltrimethoxysilane, 3-mercaptopropyltrimethoxysilane, 3-phenylpropyltrimethoxysilane, 4-amino-3,3-dimethylbutyltrimethoxysilane, vinyltrimethoxysilane, methyltrimethoxysilane, (3-triethoxysilyl)propyl]succinic anhydride, or combinations thereof;
(B) said monomeric compound comprises a hydrolyzed form of glycidylpropylmethyldiethoxysilane, 2-(3,4-epoxycyclohexyl)ethylmethyldiethoxysilane, phenyltrimethoxysilane, (1,3-di-2-propen-1-yl)-5-(([3-triethoxysilylpropyl])-(1,3,5-triazine-2,4,6 (1H,3H,5H)-trione), (methacryloxymethyl)methyl-dimethoxysilane, 2-chloroethylmethyldimethoxysilane, 3-acetoxypropylmethyldimethoxysilane, 3-mercaptopropylmethyldimethoxysilane, 3-phenylpropylmethyldimethoxysilane, 4-amino-3,3-dimethylbutylmethyldimethoxysilane, vinylmethyldimethoxysilane, dimethyl-dimethoxysilane, phenylmethyldimethoxysilane, or combinations thereof; or
(C) both (A) and (B).

6. The method of claim 1, wherein said polymer is a homopolymer, said oligomer is a homo-oligomer, or both.

7. The method of claim 1, wherein said underlayer has an average thickness of about 4.5 nm or less.

8. The method of claim 1, wherein said underlayer is a monolayer.

9. The method of claim 1, wherein said composition consists essentially of said component and a catalyst dispersed or dissolved in a solvent system.

10. The method of claim 1, wherein said composition consists essentially of said component, a catalyst, and a surfactant dispersed or dissolved in a solvent system.

11. The method of claim 1, wherein if said component is a polymer and if said monomer is 2-(3,4-epoxycyclohexyl)ethyltrimethoxysilane or a hydrolyzed form of 2-(3,4-epoxycyclohexyl)ethyltrimethoxysilane, at least one of the following is true:
said polymer has a weight-average molecular weight of less than about 2,000 Daltons;
said underlayer exhibits a % stripping of less than about 30%;
said polymer is crosslinked in said underlayer; or
said polymer is branched.

12. The method of claim 1, said stack comprising:
a substrate having a surface; and
optionally one or more intermediate layers on said surface, there being an uppermost intermediate layer on said surface, if one or more intermediate layers are present, said underlayer being on said uppermost intermediate layer, if present, or on said surface, if no intermediate layers are present.

13. The method of claim 12, said substrate comprising silicon, SiGe, SiO₂, Si₃N₄, SiON, SiCO:H, tetramethyl silate and tetramethylcyclotetrasiloxane combinations, aluminum, tungsten, tungsten silicide, gallium arsenide, germanium, tantalum, tantalum nitride, Ti₃N₄, hafnium, HfO₂, ruthenium, indium phosphide, or glass.

14. The method of claim 12, further comprising:
forming a pattern in said photoresist layer after said subjecting said photoresist layer to EUV radiation; and
transferring said pattern:
to said underlayer;
to said intermediate layers, if present; and
to said substrate.

15. The method of claim 12, wherein said stack comprises said one or more intermediate layers on said surface, said one or more intermediate layers comprising a hardmask layer.

16. The method of claim 12, wherein said photoresist layer comprises a metal.

17. The method of claim 16, wherein said photoresist layer comprises a metal oxide.

18. A structure comprising:
a substrate having a surface;
optionally one or more intermediate layers on said substrate surface, there being an uppermost intermediate layer on said substrate surface, if one or more intermediate layers are present;
an underlayer on said substrate surface, or on said uppermost intermediate layer, if present, said underlayer comprising:
(a) a polymer, an oligomer, or a mixture thereof comprising at least about 75 mol % of a monomer having an adhesion moiety at a first end of an alkyl and an Si—O moiety at a second end of said alkyl;
(b) a monomeric compound comprising an adhesion moiety at a first end of an alkyl and an Si—O moiety at a second end of said alkyl; or
(c) combinations of (a) and (b); and
a photoresist on said underlayer.

19. The structure of claim 18, wherein said photoresist is patternable at a wavelength of less than about 20 nm and/or at an exposure dose of about 5 mJ/cm² to about 100 mJ/cm².

20. The structure of claim 18, wherein said adhesion moiety is generally oriented toward said photoresist layer.

21. The structure of claim 18, wherein said Si—O moiety is generally oriented toward said uppermost intermediate layer, if present, or toward said substrate surface, if no intermediate layers are present.

22. The structure of claim 18, said substrate comprising silicon, SiGe, SiO₂, Si₃N₄, SiON, SiCO:H, tetramethyl silate and tetramethyl-cyclotetrasiloxane combinations, aluminum, tungsten, tungsten silicide, gallium arsenide, germanium, tantalum, tantalum nitride, Ti₃N₄, hafnium, HfO₂, ruthenium, indium phosphide, or glass.

23. The structure of claim 18, wherein said adhesion moiety comprises one or more of an epoxy group, an isocyanurate group, a benzene group, a carbomethoxy group, a vinyl group, an anhydride group, a carboxylic acid group, a chlorine atom, or mixtures thereof.

24. The structure of claim 18, wherein:
(I) said monomer comprises at least two Si—O moieties;
(II) said monomeric compound comprises at least two Si—O moieties; or
(III) both (I) and (II).

25. The structure of claim 18, wherein:
(A) said monomer comprises a hydrolyzed form of glycidylpropyltrimethoxysilane, 2-(3,4-epoxycyclohexyl)ethyltriethoxysilane, 2-(3,4-epoxycyclohexyl)ethyltrimethoxysilane phenyltrimethoxysilane, (1,3-di-2-propen-1-yl)-5-(([3-triethoxysilylpropyl])-(1,3,5-triazine-2,4,6 (1H,3H,5H)-trione), (methacryloxy-methyl)trimethoxysilane, 2-chloroethyltrimethoxysilane, 3-acetoxypropyltrimethoxysilane, 3-mercaptopropyltrimethoxysilane, 3-phenylpropyltrimethoxysilane, 4-amino-3,3-dimethylbutyltrimethoxysilane, vinyltrimethoxysilane, methyltrimethoxysilane, (3-triethoxysilyl)propyl]succinic anhydride, or combinations thereof;
(B) said monomeric compound comprises a hydrolyzed form of glycidylpropylmethyldiethoxysilane, 2-(3,4-epoxycyclohexyl)ethylmethyldiethoxysilane, phenyltrimethoxysilane, (1,3-di-2-propen-1-yl)-5-(([3-triethoxysilylpropyl])-(1,3,5-triazine-2,4,6 (1H,3H,5H)-trione), (methacryloxymethyl)methyl-dimethoxysilane, 2-chloroethylmethyldimethoxysilane, 3-acetoxypropylmethyldimethoxysilane, 3-mercaptopropylmethyldimethoxysilane, 3-phenylpropylmethyldimethoxysilane, 4-amino-3,3-dimethylbutylmethyldimethoxysilane, vinylmethyldimethoxysilane, dimethyl-dimethoxysilane, phenylmethyldimethoxysilane, or combinations thereof; or
(C) both (A) and (B).

26. The structure of claim 18, wherein said polymer is a homopolymer, said oligomer is a homo-oligomer, or both.

27. The structure of claim 18, wherein said underlayer has an average thickness of about 4.5 nm or less.

28. The structure of claim 18, wherein said underlayer is a monolayer.

29. The structure of claim 18, wherein if said underlayer comprises said polymer and if said monomer is derived from a hydrolyzed form of 2-(3,4-epoxycyclohexyl)ethyltrimethoxysilane, at least one of the following is true:
said underlayer exhibits a % stripping of less than about 30%;
said polymer is crosslinked in said underlayer; or
said polymer is branched.

30. The structure of claim 18, wherein said stack comprises said one or more intermediate layers on said surface, said one or more intermediate layers comprising:
a spin-on carbon layer;
a hardmask layer; or
both said spin-on carbon layer and said hardmask layer on said spin-on carbon layer.

31. The structure of claim 18, wherein said photoresist layer comprises a metal.

32. A method of hydrolyzing a silane, said method comprising combining said silane with an acid and at least one solvent and allowing said silane to hydrolyze for at least 60 minutes, wherein:
said silane is chosen from glycidylpropylmethyldiethoxysilane, 2-(3,4-epoxycyclohexyl)ethylmethyldiethoxysilane, phenyltrimethoxysilane, (1,3-di-2-propen-1-yl)-5-(([3-triethoxysilylpropyl])-(1,3,5-triazine-2,4,6 (1H,3H,5H)-trione), (methacryloxymethyl)methyl-dimethoxysilane, 2-chloroethylmethyldimethoxysilane, 3-acetoxypropylmethyldimethoxysilane, 3-mercaptopropylmethyldimethoxysilane, 3-phenylpropylmethyldimethoxysilane, 4-amino-3,3-dimethylbutylmethyldimethoxysilane, vinylmethyldimethoxysilane, dimethyl-dimethoxysilane, phenylmethyldimethoxysilane, glycidylpropyltrimethoxysilane, 2-(3,4-epoxycyclohexyl)ethyltriethoxysilane, 2-(3,4-epoxycyclohexyl)ethyltrimethoxysilane, (methacryloxy-methyl)trimethoxysilane, 2-chloroethyltrimethoxysilane, 3-acetoxypropyltrimethoxysilane, 3-mercaptopropyltrimethoxysilane, 3-phenylpropyltrimethoxysilane, 4-amino-3,3-dimethylbutyltrimethoxysilane, vinyltrimethoxysilane, methyltrimethoxysilane, (3-triethoxysilyl)propyl]succinic anhydride, or combinations thereof; and
said acid is chosen from maleic acid, nitric acid, hydrochloric acid, acetic acid, sulfuric acid, or combinations thereof.

33. The method of claim 32, wherein said combining forms a reaction mixture and:
said silane is chosen from glycidylpropylmethyldiethoxysilane, 2-(3,4-epoxycyclohexyl)ethylmethyldiethoxysilane, phenyltrimethoxysilane, (1,3-di-2-propen-1-yl)-5-(([3-triethoxysilylpropyl])-(1,3,5-triazine-2,4,6 (1H,3H,5H)-trione), (methacryloxymethyl)methyl-dimethoxysilane, 2-chloroethylmethyldimethoxysilane, 3-acetoxypropylmethyldimethoxysilane, 3-mercaptopropylmethyldimethoxysilane, 3-phenylpropylmethyldimethoxysilane, 4-amino-3,3-dimethylbutylmethyldimethoxysilane, vinylmethyldimethoxysilane, dimethyl-dimethoxysilane, phenylmethyldimethoxysilane, or combinations thereof;
said silane is combined into said reaction mixture at a level of about 1% to about 15% by weight based on the total weight of the reaction mixture; and
said acid is combined into said reaction mixture at a level of about 0.001 mol % to about 0.1 mol %, based on the total moles of silane.

34. The method of claim 32, wherein said combining forms a reaction mixture and:
said silane is chosen from glycidylpropyltrimethoxysilane, 2-(3,4-epoxycyclohexyl)ethyltriethoxysilane, 2-(3,4-epoxycyclohexyl)ethyltrimethoxysilane phenyltrimethoxysilane, (1,3-di-2-propen-1-yl)-5-(([3-triethoxysilylpropyl])-(1,3,5-triazine-2,4,6 (1H,3H,5H)-trione), (methacryloxy-methyl)trimethoxysilane, 2-chloroethyltrimethoxysilane, 3-acetoxypropyltrimethoxysilane, 3-mercaptopropyltrimethoxysilane, 3-phenylpropyltrimethoxysilane, 4-amino-3,3-dimethylbutyltrimethoxysilane, vinyltrimethoxysilane, methyltrimethoxysilane, (3-triethoxysilyl)propyl]succinic anhydride, or combinations thereof;
said silane is combined into said reaction mixture at a level of about 1% to about 25% by weight based on the total weight of the reaction mixture;
said acid is combined into said reaction mixture at a level of about 0.5 equivalents to about 20 equivalents to the total monomers; and
said reaction is carried out at a temperature of about 20° C. to about 100° C.
